(19) United States
(12) Patent Application Publication
(10) Pub. No.: US 2014/0141099 A1
(43) Pub. Date: May 22, 2014

(54) DRUG DISCOVERY METHODS

(71) Applicant: Vertex Pharmaceuticals Incorporated, Cambridge, MA (US)

(72) Inventors: Julian Golec, Faringdon (GB); John Pollard, Abingdon (GB); James Westcott, Witney (GB); Hayley Binch, Encinitas, CA (US); Michael Mortimore, Burford (GB); Daniel Robinson, Abingdon (GB)

(21) Appl. No.: 13/930,052

(22) Filed: Jan. 27, 2014

Related U.S. Application Data
(63) Continuation of application No. 12/595,878, filed on Oct. 14, 2009, now abandoned.
Application No. 12/595,878 is a continuation of application No. PCT/US08/60635, filed on Apr. 17, 2008.
(60) Provisional application No. 60/912,271, filed on Apr. 17, 2007.

(51) Int. Cl. C07D 403/12; C12Q 1/48; A61K 45/06; A61K 31/506; C07D 401/14

(52) U.S. Cl. CPC C07D 403/12; A61K 31/506; C07D 401/14; A61K 45/06; C12Q 1/485
USPC 424/649; 544/295; 514/252.19; 544/317; 514/274; 514/154; 514/234.8; 514/34; 435/184; 435/15

(57) ABSTRACT

The present invention relates to drug discovery methods, particularly methods for assaying compounds for activity as Aurora kinase inhibitors. This invention also relates to a pharmacophore describing compounds that are able to promote a conformational change in the protein AuroraB and whose binding constant for the two-step process is given as Ki*. Finally, this invention also relates to compounds having the features of the pharmacophore.

### CROSS-REFERENCE TO RELATED APPLICATIONS

This application is a continuation of U.S. Non-provisional patent application Ser. No. 12/595,878, filed on Oct. 14, 2009, which is a continuation application of International Patent Application No. PCT/US2008/060635, filed Apr. 17, 2008, which claims the benefit under 35 U.S.C. §119, of U.S. Provisional Patent Application No. 60/912,271, filed Apr. 17, 2007, the entire contents of these applications are hereby incorporated by reference.

### BACKGROUND

1. Field of the Invention

The present invention relates to methods of identifying compounds that are Aurora kinase inhibitors.

2. Background of the Related Art

The need for improved Aurora kinase inhibitors is well known. Cancer is a compelling human medical problem. Thus, there is a need for more effective Aurora kinase inhibitors. Such inhibitors would have therapeutic potential as anticancer agents.

### SUMMARY OF THE INVENTION

This invention addresses the above problems by providing novel drug discovery methods and compounds identified by those methods. Applicants' method is based on the structural analysis of Aurora kinases and the binding kinetics of compounds that inhibit Aurora kinases. This invention provides methods for assaying compounds for activity as Aurora kinase inhibitors. This invention also provides a pharmacophore describing compounds that are able to promote a conformational change in the protein AuroraB and whose binding constant for the two-step process is given as Ki*. This invention also provides compounds having the features of the pharmacophore.

Other features and advantages of the invention will become apparent from the following detailed description. It should be understood, however, that the detailed description and the specific examples, while indicating preferred embodiments of the invention, are given by way of illustration only, because various changes and modifications within the spirit and scope of the invention will become apparent to those skilled in the art from this detailed description.

### BRIEF DESCRIPTION OF THE DRAWINGS

The following drawings form part of the present specification and are included to further illustrate aspects of the present invention. The invention may be better understood by reference to the drawings in combination with the detailed description of the specific embodiments presented herein.

FIG. 1A depicts one embodiment in accordance with this invention.

FIG. 1B illustrates the role of Aurora A, Aurora B, and Aurora C in regulation of mitosis.

FIG. 1C depicts a dialogue box describing the rationale for targeting Aurora kinases as drug discovery targets.

FIG. 1D illustrates a first-in-class pan-Aurora inhibitor.

FIG. 1E depicts a chart showing the Ki and anti-proliferative effects of MK-0457.

FIG. 1F depicts a chart showing the variation in anti-proliferative activity between Aurora kinase inhibitors MK-0457, VRT-426, and VRT-960.

FIG. 1G depicts a graph the kinetic profile of Aurora kinase inhibitor VRT-960.

FIG. 1H depicts graphs of the kinetic profiles of Aurora kinase inhibitors VRT-960 and MK-0457.

FIG. 1I depicts graphs of the kinetic profiles of Aurora kinase inhibitors MK-0457, VRT-426, and VRT-960.

FIG. 1J illustrates further graphs of the kinetic profile of VRT-426.

FIG. 1K depicts the graphs of FIG. 1J with a comparison chart illustrating the differences in the kinetics between Aurora kinase inhibitors MK-0457, VRT-960, and VRT-426.

FIG. 1L depicts two conformations of the Aurora enzyme.

FIG. 1M depicts the conformation of the Aurora enzyme when MK-0457 is bound to the active site.

FIG. 1N discloses a diagram indicating the Aurora enzyme has a moderate affinity for VRT-426 when the enzyme is in the closed/inactive conformation.

FIG. 1O discloses a diagram indicating the Aurora enzyme has a strong affinity for VRT-426 when the enzyme is in the open/active conformation.

FIG. 1P illustrates a chart showing the effects sterics have on the binding properties between the enzyme and the Aurora inhibitor.

FIG. 1Q illustrates a graph showing the anti-proliferative properties of VRT-426, MK-0457, and VRT-960 on Hct116 colon cells.

FIG. 1R illustrates three graphs showing the effects VRT-426, MK-0457, and VRT-960 have on the cell cycle.

FIG. 1S illustrates two graphs showing the anti-tumor activity of VRT-426 in vivo.

FIG. 1T illustrates a dialog box providing a summary of the potential benefits of Aurora kinase inhibitor MK-0457.

FIG. 1U illustrates a dialog box providing an additional summary of the benefits of Aurora kinase inhibitor MK-0457.

FIG. 2A depicts schemes for preparing compounds of this invention.

FIG. 2B depicts an additional scheme for preparing compounds of this invention.

FIG. 2C depicts another scheme for preparing compounds of this invention.

FIG. 2D depicts a scheme for preparing intermediates of this invention.

FIG. 2E depicts another scheme for preparing intermediates of this invention.

FIG. 2F depicts yet another scheme for preparing intermediates of this invention.

FIG. 3 depicts a return of activity assessed by determining the observed rate of change (k_{obs}) of the reaction progress curve. k_{obs} plotted, as a function of inhibitor concentration, to a two-step binding model.

FIG. 4 depicts demonstration in an animal model that once a week dosing of compound 2, a compound with a favorable Ki/Ki* ratio, resulted in very good tumor growth inhibition.

FIG. 5 depicts a graph of rapid binding kinetics turnover of substrate.

FIG. 6 shows a graph depicting an inhibitor displaying slow binding kinetics, turnover of substrate to product.

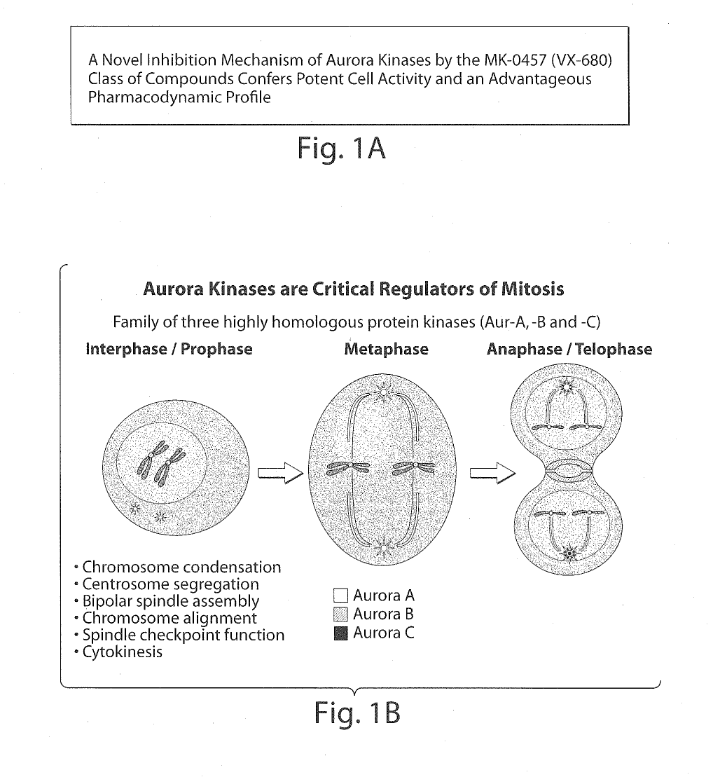

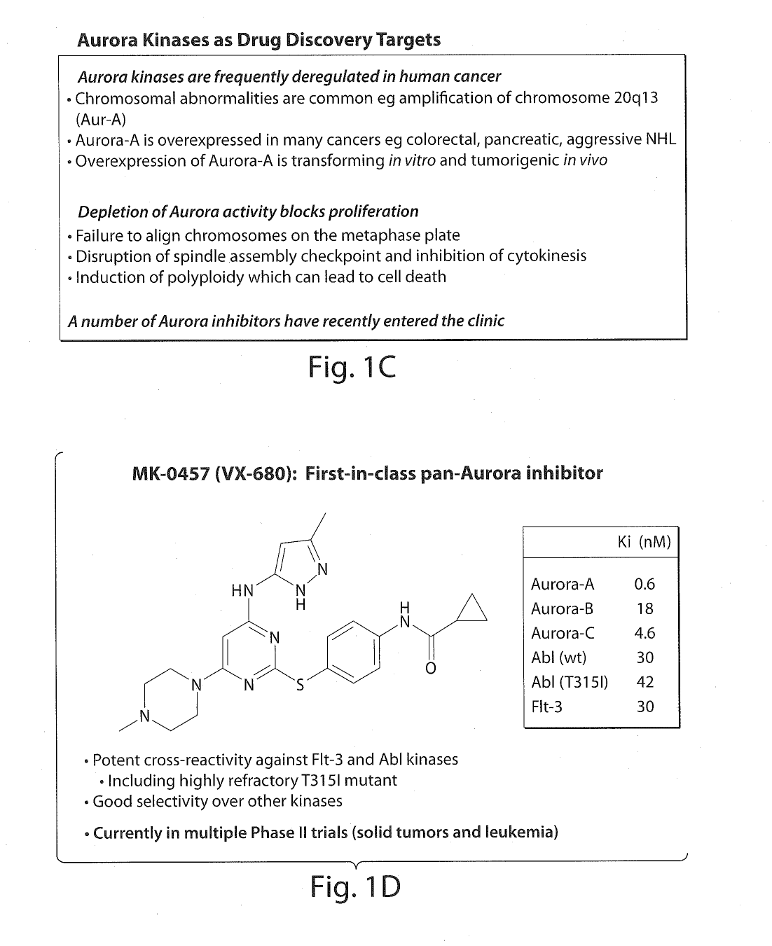

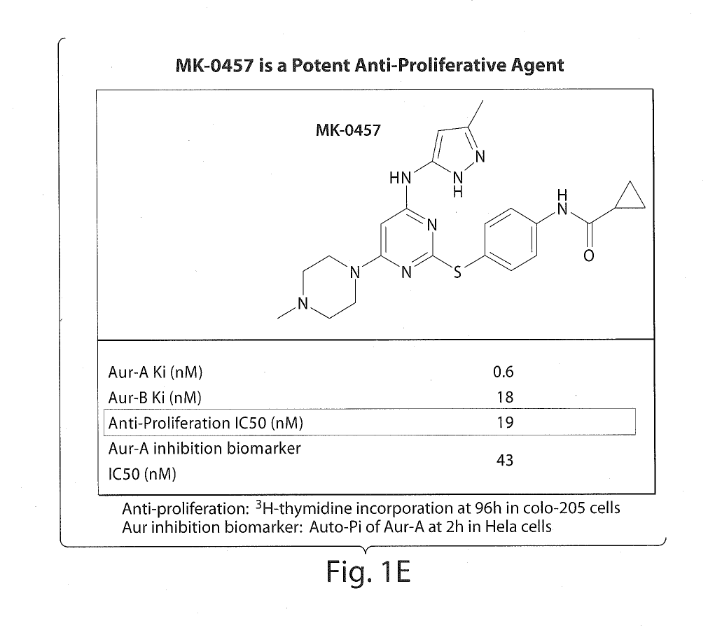

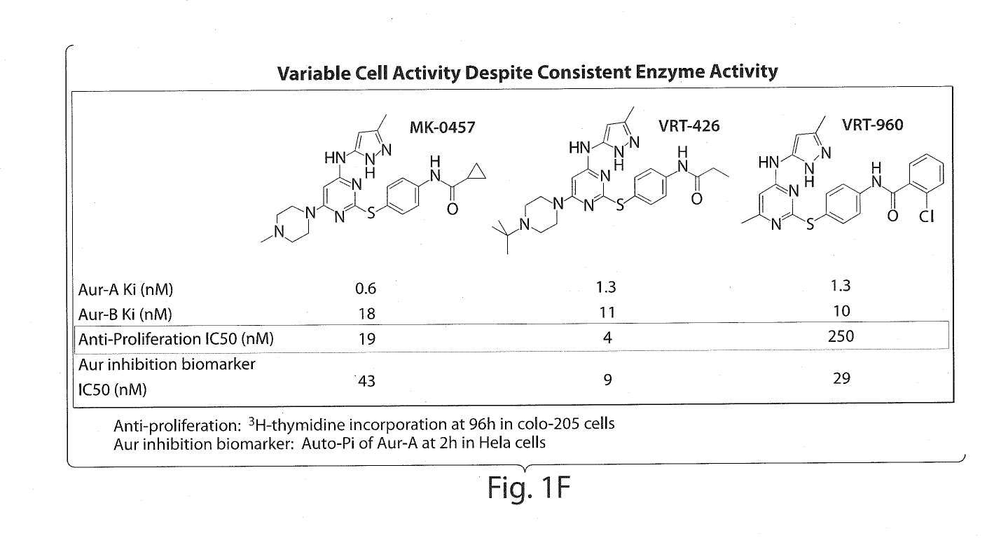

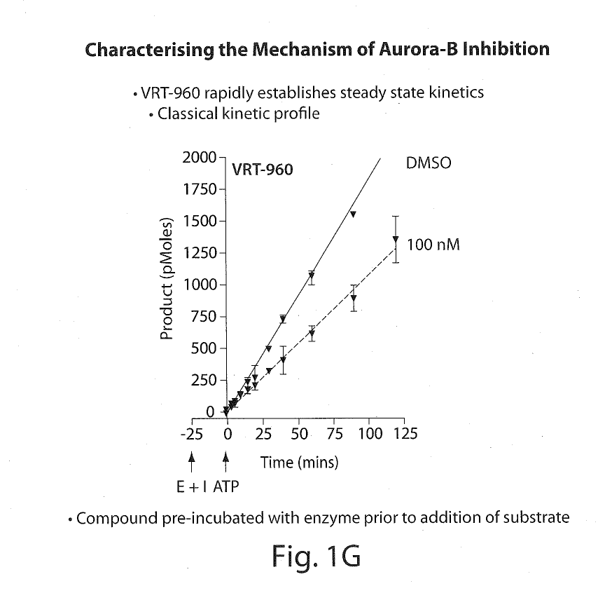

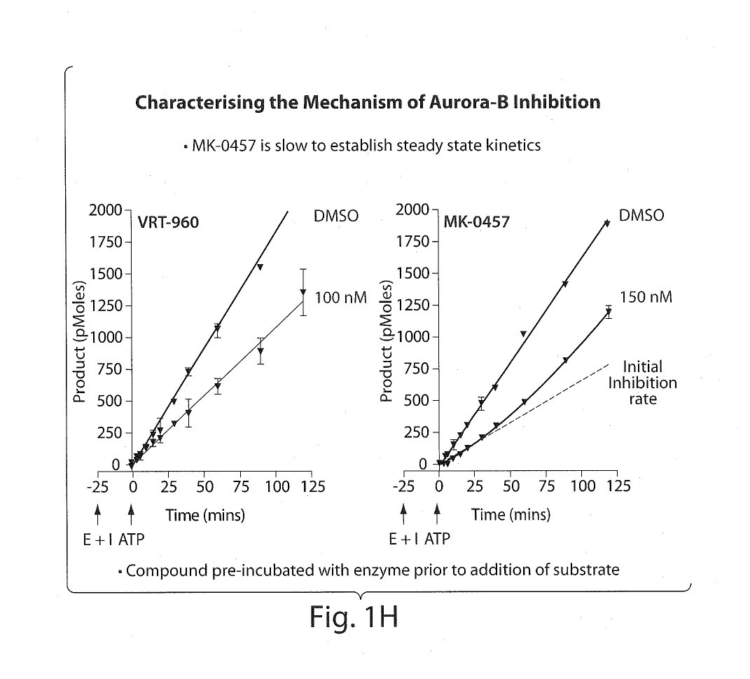

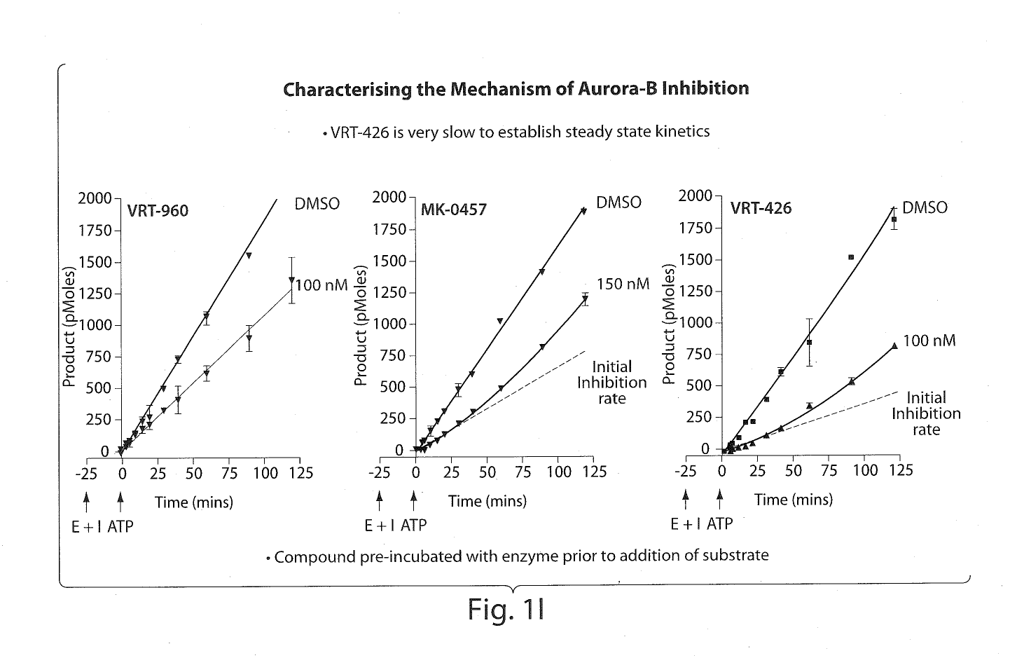

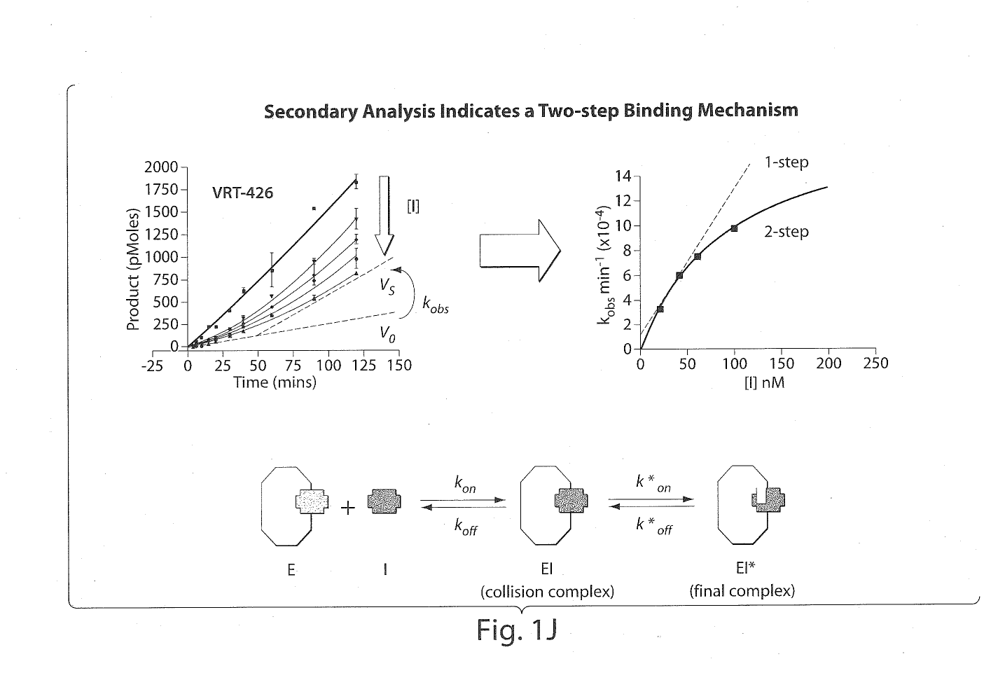

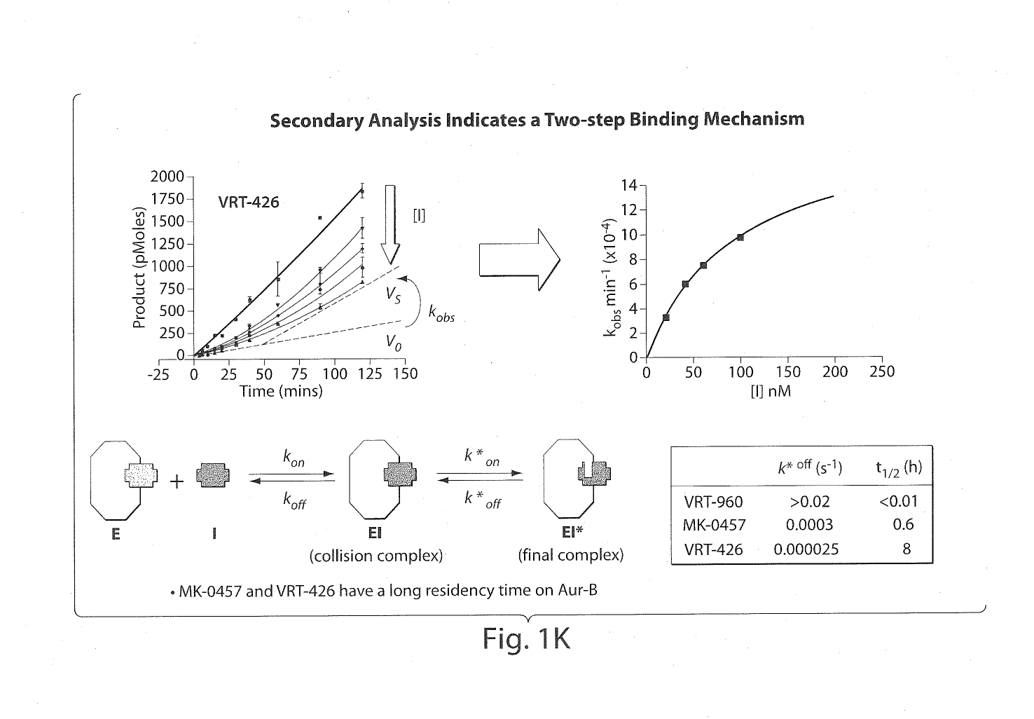

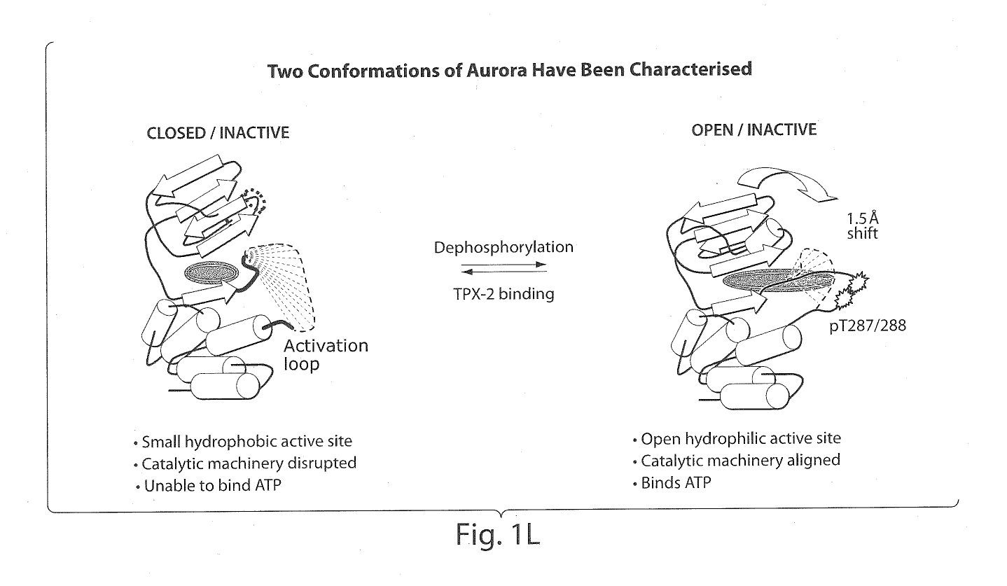

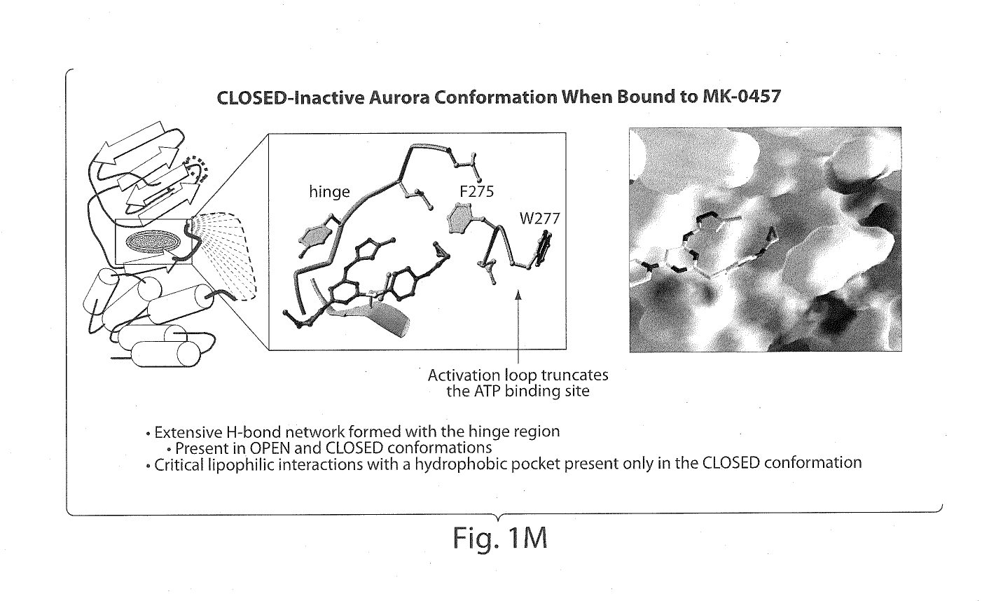

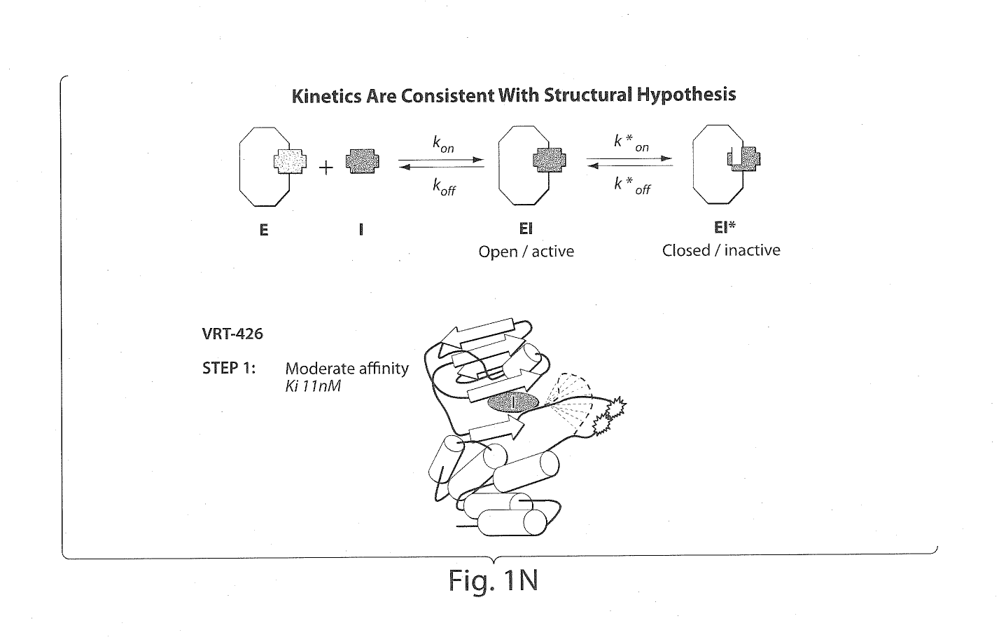

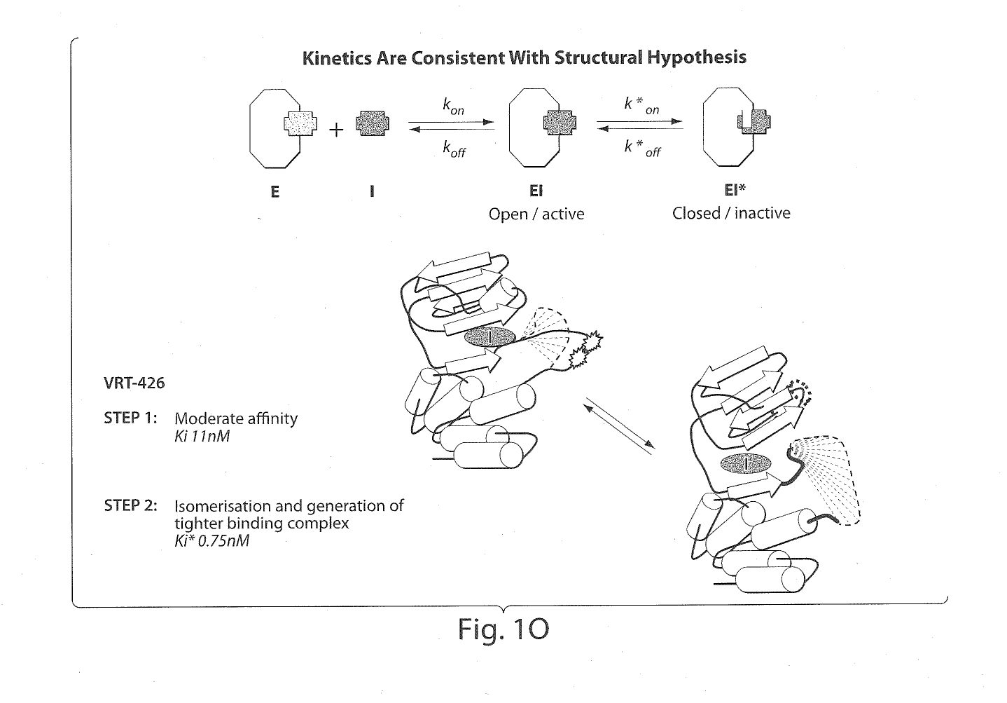

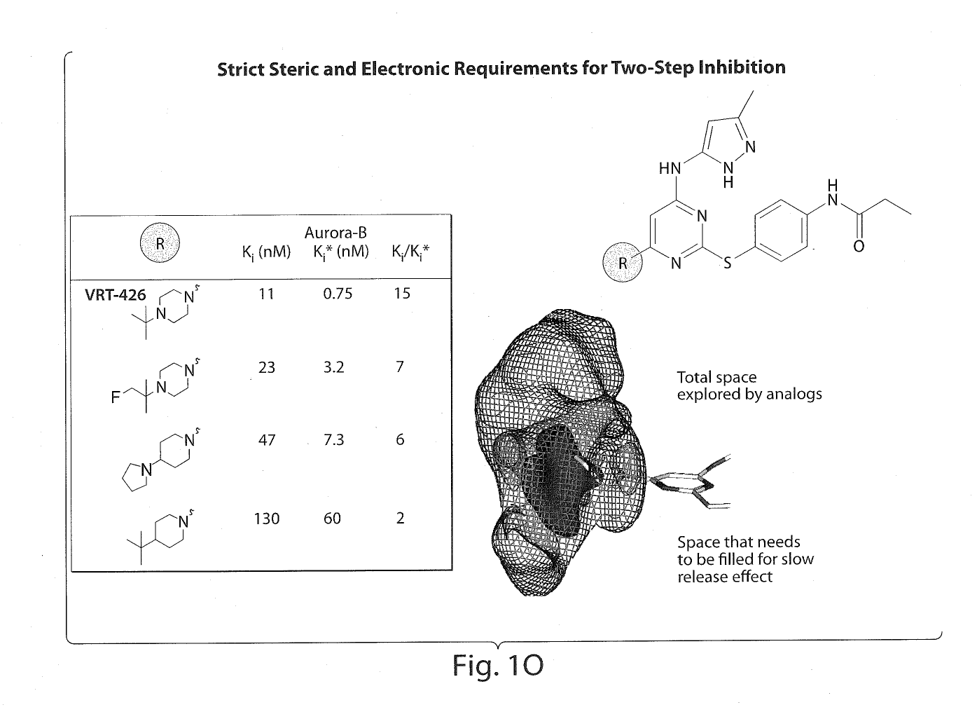

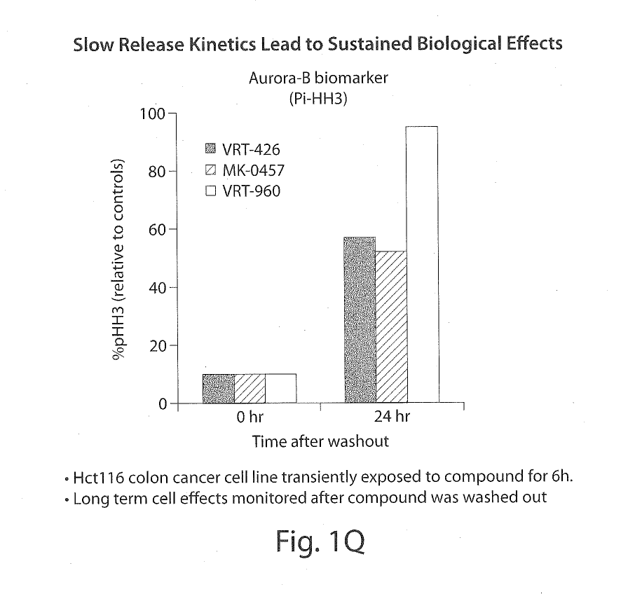

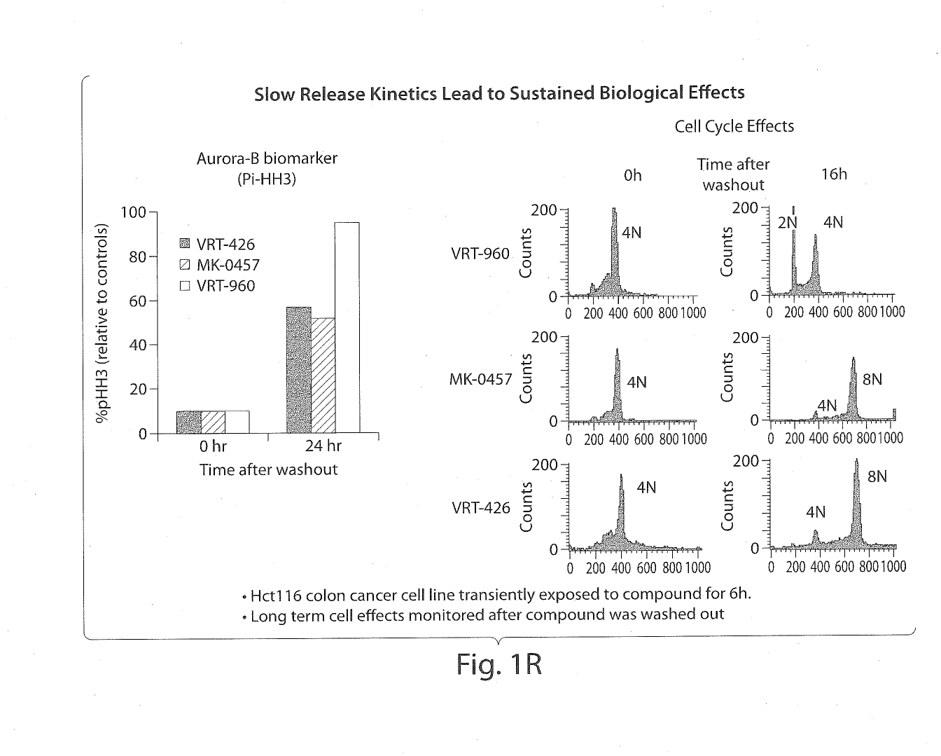

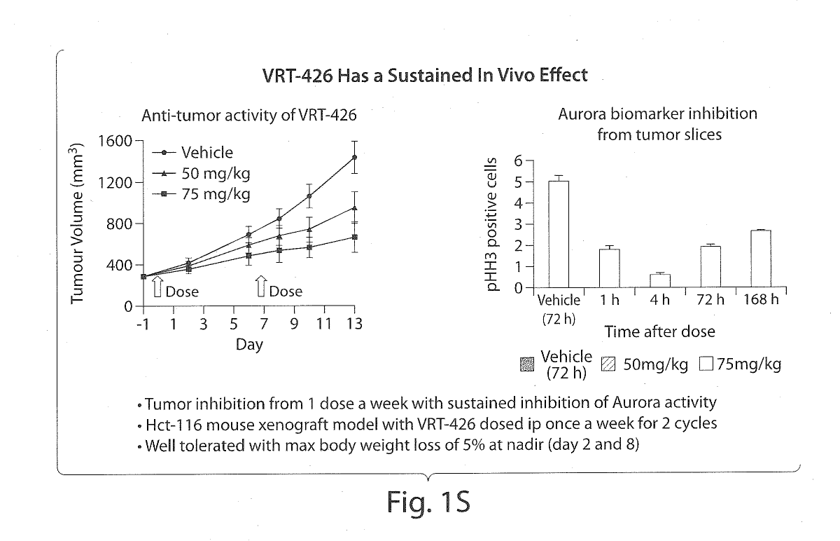

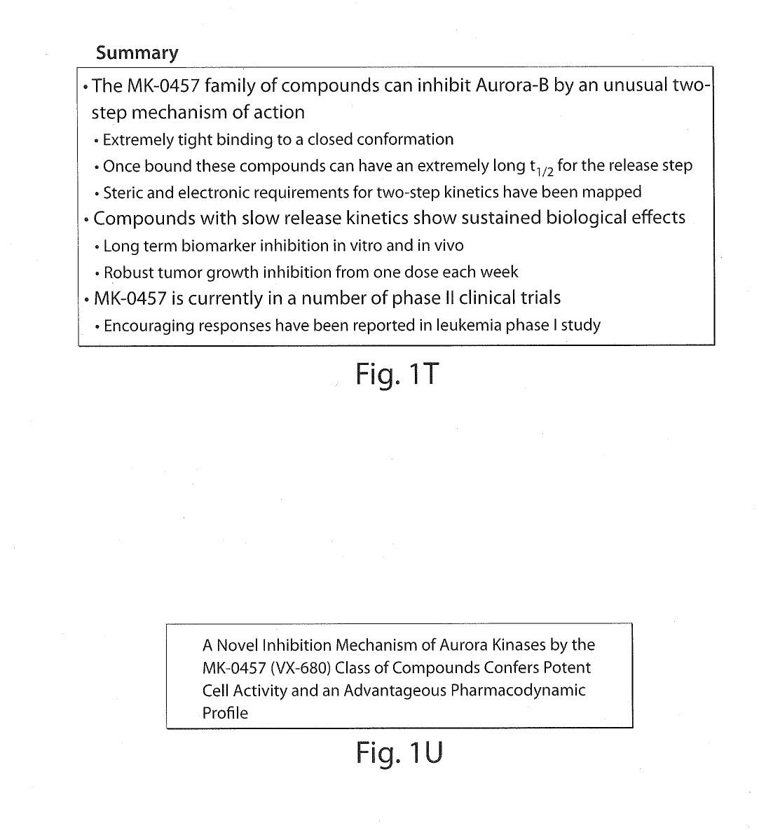

### DETAILED DESCRIPTION

In one embodiment, this invention provides methods for assaying compounds for activity as Aurora kinase inhibitors.

In another embodiment, this invention provides a pharmacophore describing compounds that are able to promote a conformational change in the protein AuroraB and whose binding constant for the two-step process is given as Ki*.

In another embodiment, this invention provides compounds having the features of the pharmacophore.

Two distinct conformations for Aurora kinase are known. In the closed/inactive conformation, there is a small hydrophobic active site, the catalytic machinery is disrupted, and the kinase is unable to bind ATP. In the open/active form, there is a larger, hydrophilic active site, the catalytic machinery is aligned, and the kinase binds ATP.

Certain compounds bind to the closed/inactive Aurora conformation. Applicants have determined that there is an extensive H-bond network formed with the hinge region, which is present in both the open and closed conformations. There are also critical lipophilic and hydrogen bond donor/acceptor interactions with a hydrophobic pocket present in only the closed conformation.

Accordingly, one embodiment of this invention provides a method of identifying compounds that have these critical lipophilic or hydrogen bond acceptor interactions with the hydrophobic pocket in the closed conformation.

Another embodiment provides a method of identifying compounds that have lipophilic or hydrogen bond acceptor interactions with the hydrophobic pocket in the closed conformation.

These compounds can be identified according to methods known to one of skill in the art (See, e.g., Khedkar S A, Malde A K, Coutinho E C, Srivastava S. Pharmacophore modeling in drug discovery and development: an overview. Med Chem. 2007 March; 3(2):187-97. PMID: 17348856; See also, Güner O F. History and evolution of the pharmacophore concept in computer-aided drug design. Curr Top Med Chem. 2002 December; 2(12):1321-32. PMID: 12470283). Examples of computer programs that may be used include, but are not limited to, Catalyst (Accelrys Software Company, USA), MOE (Chemical Computing Group, Canada) and Phase (Schrodinger Inc., USA).

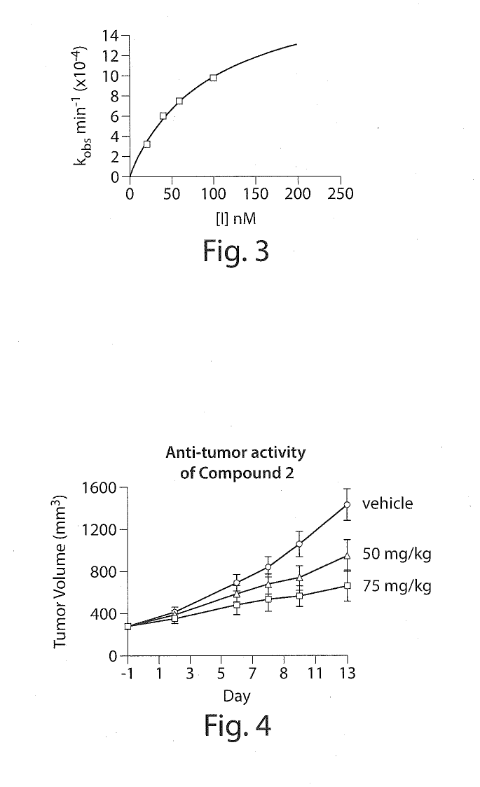

Inhibition kinetics indicates an unusual mechanism of inhibition. In particular, certain compounds exhibit a time-dependent tight binding inhibition. This mechanism is observed upon pre-incubation of a compound in the presence of enzyme and in the absence of substrate (ATP). ATP is added and the return of activity is assessed by determining the observed rate of change (k_{obs}) of the reaction progress curve. k_{obs} is plotted, as a function of inhibitor concentration, to a two-step binding model that is depicted in FIG. 3.

Applicants have discovered that for compounds that display a two-stop binding mechanism with slow, tight-binding kinetics, the Ki* value is a much better predictive tool for cell potency than is Ki. In some embodiments, such compounds have a strong pharmacodynamic profile, resulting in long term cell activity that would allow for shorter dosing regimens in vivo. A typical dosing regimen for Aurora inhibitors in animal models is, e.g., at least once a day dosing. In one embodiment, applicants' invention allows for selecting compounds that may be dosed less than once a day. For example, applicants have demonstrated in an animal model that once a week dosing of compound 2, a compound with a favorable Ki/Ki* ratio, resulted in very good tumor growth inhibition, in FIG. 4.

Without being bound by theory, dosing twice a day would be typical for compounds displaying normal binding kinetics. Accordingly, applicants' invention provides a compound where one dose of a compound results in long-lasting effects in vivo.

A critical question in any drug discovery effort is which assay to use to select compounds for further testing and/or further development. Once an assay is selected and results obtained, a further critical question is how to use those results to select a compound of interest (e.g., one to investigate further; one that will be a successful drug). These uncertainties lead to problems in effectively and efficiently conducting drug discovery.

Applicants' invention addresses these problems by providing assays and a method of using the assays to conduct drug discovery.

Applicants have identified the importance of certain measurements (or comparisons) in the drug discovery process. Traditional measurements, such as Ki and/or IC50, although useful, may not be sufficient for fully evaluating an inhibitor. Such measurements may, however, be used in conjunction with this invention.

An important aspect of this invention is the time an inhibitor remains associated with the target after each time it binds (as express by k_{off} or t1/2 of the target-inhibitor complex). In particular, applicants' invention provides that the time a compound remains associated with a target after each time that it binds to the target correlates with the effectiveness that the compound inhibits the target.

Ki* as used herein is related to the overall binding affinity of a compound to Aurora kinase where the mechanism of inhibition occurs as a two step binding process. With this mechanism the second step of the binding process forms a high affinity complex of the inhibitor to an isomerized or conformationally modified form of the enzyme herein termed as the “closed conformation.” In accordance with this invention, potency is driven by a high affinity for the closed form as measured by Ki*. Long residency times may have a pharmacodynamic advantage.

The pocket where the position 6 group (e.g., alkyl-piperazine; see Formula I) binds in the Aurora structure is disordered. Therefore, it has been difficult to obtain structural information in this region of interest that seems to be important for driving slow and tight-binding. There is therefore a need for methods to evaluate this region of interest. Applicants' invention addresses these problems by providing such methods.

In one embodiment, this invention provides a selection criteria for drug discovery. Steps involved in a method of this invention may optionally comprise:

Identifying an inhibitor or a subset of inhibitors to be evaluated in accordance with this invention;
Determining Ki;
Determining Ki*;
Selecting a compound that has a Ki/Ki* of greater than 1.

In a preferred embodiment, the compound has a Ki/Ki* of greater than 3.

In some embodiments, inhibitors that make lipophilic or hydrogen bond acceptor interactions with a hydrophobic pocket of the Aurora kinase (preferably Aurora B) in the closed conformation are identified. In some embodiments, inhibitors having critical lipophilic or hydrogen bond acceptor interactions with a hydrophobic pocket of the Aurora kinase (preferably Aurora B) in the closed conformation are identified.

Some embodiments provide a method for selecting a compound having activity as an Aurora inhibitor comprising the step of identifying an inhibitor or a subset of inhibitors having critical lipophilic interactions with the hydrophobic pocket of the Aurora kinase in the close conformation.

In some embodiments, said inhibitors are Aurora kinase inhibitors, preferably Aurora B kinase inhibitors.

Accordingly, another embodiment provides a method for selecting an Aurora B inhibitor that has certain drug-like properties (e.g., cell activity, pharmacodynamic properties, in vivo efficacy) comprising steps a) or b):

a) Identifying an inhibitor that
1) makes hydrogen bonds to the hinge region of the Aurora B kinase;
2) makes lipophilic interactions with a first hydrophobic pocket of the Aurora B kinase, wherein said first hydrophobic pocket is the space occupied by the S-phenyl moiety of a compound of formula I; and
3) makes lipophilic or hydrogen bond interactions with a second hydrophobic pocket of the Aurora B kinase in the closed conformation; wherein said second hydrophobic pocket is the space occupied by position 6 of compounds of formula I:

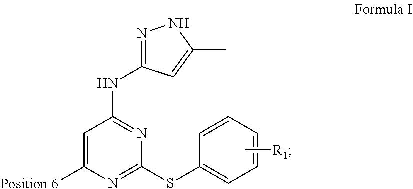

wherein

R¹ is —NHC(O)R², OR³; or two R¹ groups, taken together, form a fused phenyl ring;

### R² is CH₂CH₃, CH₂CF₃, CH₂CH₂CF₃,

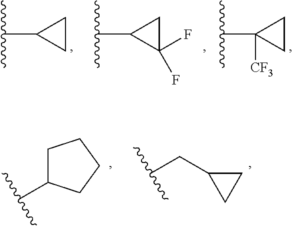

or phenyl optionally substituted with halo, CF₃, or C₁₋₃alkyl; and
R³ is C₁₋₄alkyl, C₃₋₆cycloalkyl;

b) Determining Ki;
Determining Ki*; and
Selecting a compound if it has a Ki/Ki* of greater than 3.

In some embodiments, step a) is used. In other embodiments, step b) is used. In yet other embodiments, both steps a) and b) are used. In some embodiments, compounds are selected if they meet the requirements of one of more of steps a) and b).

As would be known by one of skill in the art, the pyrazole of formula I can be replaced by other Aurora hinge binders, such as those described in WO2002/057259, WO2004/000833, WO 2007/056221, WO 2007/056163, or WO 2007/056164.

In some embodiments, the pyrazole of formula I can be replaced by

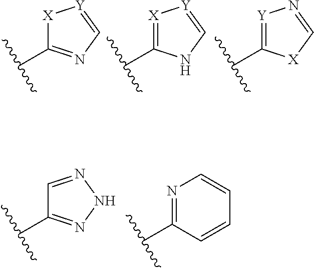

wherein X is sulfur, oxygen, or NR^{2′} and Y is nitrogen or CR²; wherein
R² is as defined according to the definition of R² in WO2002/057259, WO2004/000833, WO 2007/056221, WO 2007/056163, or WO 2007/056164.

In some embodiments, R² is C₁₋₆alkyl, C₃₋₈cyclopropyl, O(C₁₋₆alkyl), CO₂(C₁₋₆alkyl), oxo, halo, CN, or phenyl. In some embodiments, R² is C₁₋₆alkyl or C₃₋₈cyclopropyl.

R^{2′} is H or C₁₋₆alkyl;

In yet other embodiments, R² and R^{2′} are optionally taken together to form a optionally substituted 5-7 membered, partially unsaturated or fully unsaturated ring having zero to two ring heteroatoms independently selected from nitrogen, oxygen, or sulfur.

In some embodiments, the pyrazole of formula I can be replaced by

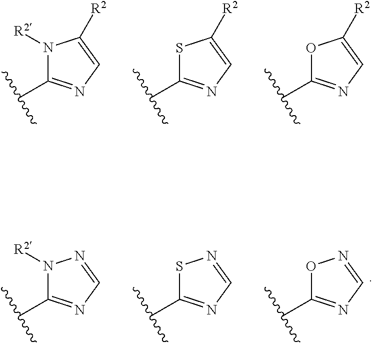

In yet other embodiments, the pyrazole of formula I can be replaced by

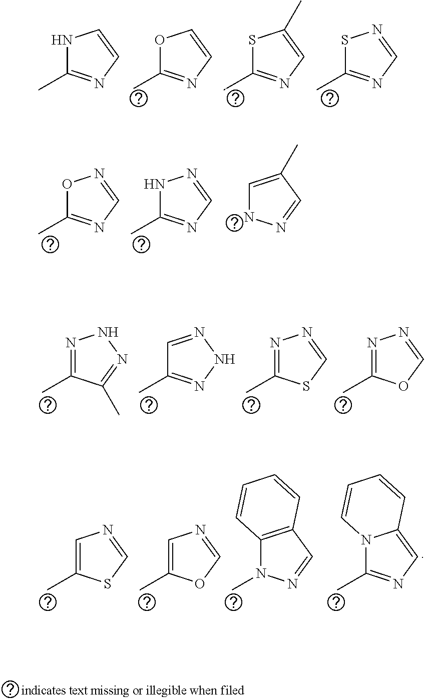

In some embodiments, the S-phenyl moiety of a compound of formula I can be replaced by S-heteroaryl, wherein heteroaryl is selected from an 8-12 membered bicyclic heteroaryl containing 1-5 heteroatoms selected from O, N, and S. Examples include, but are not limited to, benzimidazole, indazole, or imidazopyridine ring.

In some embodiments, the core pyrimidine ring can be replaced by another core scaffold that allows the pyrazole moiety, the position 6 moiety, and the S-phenyl moiety to be in the same positions as they are with respect to the pyrimidine ring in Formula I. Examples of replacement include, but are not limited to, triazine, pyridine, and alternate pyrimidine cores.

In some embodiments, said second hydrophobic pocket is the space occupied by position 6 of compounds of formula I:

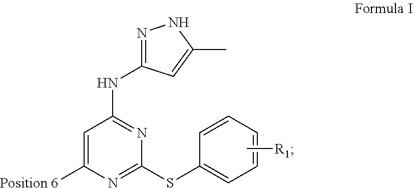

wherein

R¹ is —NHC(O)R², OR³; or two R¹ groups, taken together, form a fused phenyl ring;

### R² is CH₂CH₃, CH₂CF₃, CH₂CH₂CF₃,

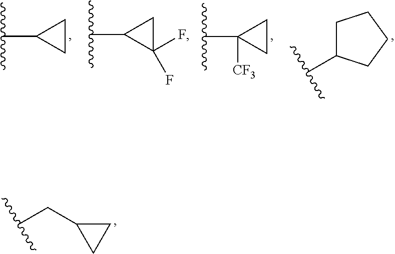

or phenyl optionally substituted with halo, CF₃, or C₁₋₃alkyl; and

R³ is C₁₋₄alkyl, C₃₋₆cycloalkyl;

Another embodiment provides a method for selecting an Aurora B inhibitor that has certain favorable properties (e.g., cell activity, pharmacodynamic properties, in vivo efficacy) comprising the step of

Determining Ki of said inhibitors;

Determining Ki* of said inhibitors; and

Selecting the inhibitor if it has a Ki/Ki* of greater than 1 (preferably greater than 3).

Another embodiment provides methods for selecting compounds that have favorable drug-like properties, such as cell activity, pharmacodynamic properties, and in vivo efficacy. In some embodiments, applicants' methods select for compounds that have higher cell penetration, improved pharmacodynamic properties, or better in vivo efficacy than compound A (described herein). In some embodiments, applicants' methods select for compounds that have a shorter dosing regimen than that of compound A.

In some embodiments, applicants provide a method for selecting compounds that promote a conformational change in the protein Aurora B.

In other embodiments, inhibitors are selected if they have a Ki/Ki* of greater than 1, preferably greater than 3. In other embodiments, inhibitors are selected if they make lipophilic or hydrogen bond acceptor interactions with a hydrophobic pocket of the Aurora kinase in the closed conformation. In yet other embodiments, inhibitors are selected if they 1) make lipophilic or hydrogen bond acceptor interactions with a hydrophobic pocket of the Aurora kinase in the closed conformation and 2) if they have a Ki/Ki* of greater than 1, preferably greater than 3—i.e. compounds are only selected if they meets the requirements of 1) making the lipophilic or hydrogen acceptor interactions and 2) have a Ki/Ki* value of >1, preferably greater than 3.

In some embodiments, identifying the inhibitor that makes lipophilic or hydrogen bond interactions is done by comparing the three-dimensional structure of a test compound with the three-dimensional structure of a pharmacophore based on formula I, wherein the pharmacophore comprises a lipophilic group and a lone pair of electrons extending the 6-position of compounds of formula I wherein the centre of the lipophilic group (hydrophobe) extends from the 6-position by 4-8 Å and lie above or below the plane by 0-4 Å; the position of the lone-pair of electrons extends from the 6-position by 3-8 Å and lies above or below the plane by 0-4 Å; the volume that the hydrophobe occupies is 70-120 Å³; and selecting the test compound if the test compound conforms to the features of the pharmacophore.

In yet other embodiments, identifying the inhibitor that makes lipophilic or hydrogen bond interactions is done by
i. preparing an atomic model of the second hydrophobic pocket of the Aurora kinase by identifying a pharmacophore reflecting distances between the 6-position of compounds of formula I, a lipophilic group, and a lone pair of electrons;
ii. screening said pharmacophore against a library of atomic models of small molecules.

In some embodiments, said test compound is a compound of formula I.

In some embodiments, the methods comprise a step of contacting the test compound with an enzyme, such as Aurora kinase (in some embodiments, Aurora B kinase).

In other embodiments, the methods comprise contacting the test compound with an enzyme, such as Aurora kinase (in some embodiments, Aurora B kinase), to measure the ability of the compound to inhibit the activity of the enzyme. In yet other embodiments, the methods comprise contacting the test compound with an enzyme, such as Aurora kinase (in some embodiments, Aurora B kinase), to evaluate the ability of the compound to inhibit the activity of the enzyme.

In some embodiments, said small molecules are Aurora kinase inhibitors.

Another embodiment provides a method for carrying out an Aurora enzyme assay for measuring Ki*.

This drug discovery method facilitates the development and design of drugs optimized for various drug properties (e.g., better solubility, improved pK, affinity for a particular ligand, better absorption in vivo) that still retaining good pharmacodynamic properties.

In some embodiments, Aurora kinase refers to Aurora B kinase.

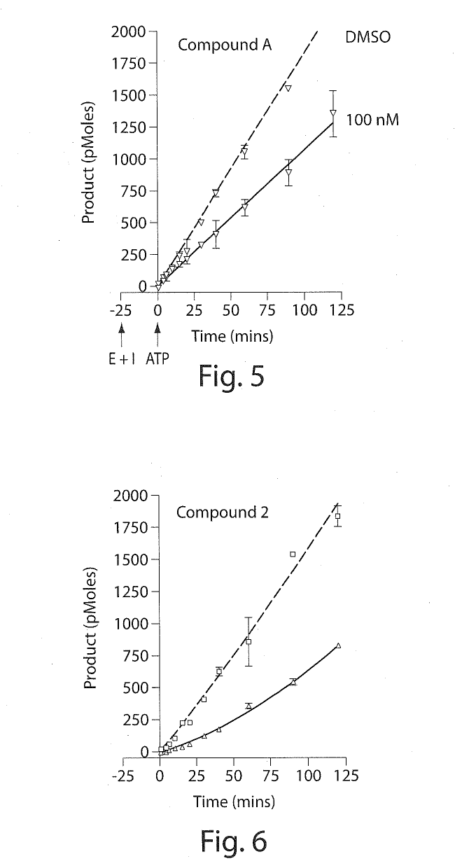

A classic reversible inhibitor will be expected to display rapid binding kinetics turnover of substrate to product would be represented as a linear curve (see FIG. 5).

In the case of an inhibitor displaying slow binding kinetics, turnover of substrate to product would be represented by a non-linear curve describing return of enzyme activity with time (see FIG. 6).

Different Ki and Ki* values imply a 2-step binding mechanism. A 2-step binding mechanism in turn implies a long residency time of a compound on an enzyme (particularly with a low Ki*). Applicants have provided two methods for measuring Ki* (see Example 1 and Example 2). Measuring Ki/Ki* is a surrogate for measuring the residency time or koff. In one method, a series of measurements is taken (Example 1). In the other method, the series of measurements is avoided by taking readings at two points of time and extrapolating the measurements (Example 2).

Applicants' method provides for pre-incubation of a test compound and an Aurora kinase (in one embodiment, Aurora B) followed by a rapid dilution of the assay mixture. Kinetics are then determined over a time-course.

Without being bound by theory, applicants' preincubation step allows a binding equilibrium to be established between enzyme and inhibitor. Dilution of the enzyme-inhibitor complex into a buffer containing substrate allows monitoring of the substrate turnover to product and also return of enzyme activity. This allows for identifying compounds with a slow off rate and long residency on the kinase.

Applicants' assay may be used to identify or evaluate drug-like molecules. Preferably, applicants' assay is used to identify or evaluate molecules with favorable pharmacodynamic profiles. Accordingly, this invention also provides a method for designing an Aurora B kinase inhibitor by using a pharmacophore, such as the pharmacophore described below.

This invention provides a pharmacophore that has been developed using compounds that are illustrated using an example based upon a compound of formula I wherein the variables are as defined herein.

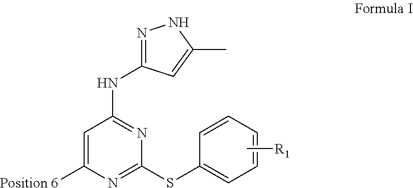

The pharmacophore describes the positioning of a lipophilic group and a lone pair of electrons extending from the 6-position of the pyrimidine ring in the compounds of formula I.

The centre of the lipophilic group (hydrophobe) should extend from the 6-position by 4-8 Å, preferably 4-6 Å, and more preferably 4-5 Å and lie above or below the plane by 0-4 Å, preferably 0-2 Å. The volume that the hydrophobe should occupy is 70-120 Å³, preferably 80-110 Å³, more preferably 80-100 Å³.

The position of the lone-pair of electrons should extend from the 6-position by 3-8 Å, preferably 3-6 Å, and more preferably 4-5 Å and lie above or below the plane by 0-4 Å, preferably 0-2 Å.

In some embodiments, the centre of the lipophilic group (hydrophobe) extends from the 6-position by 4-8 Å and lie above or below the plane by 0-4 Å; the position of the lone-pair of electrons extends from the 6-position by 3-8 Å and lies above or below the plane by 0-4 Å; the volume that the hydrophobe occupies is 70-120 Å³.

In other embodiments, the hydrophobe extends from the 6-position by 4-6 Å and lie above or below the plane by 0-2 Å; the position of the lone-pair of electrons extends from the 6-position by 3-6 Å and lies above or below the plane by 0-2 Å; the volume that the hydrophobe occupies is 80-110 Å³.

In yet other embodiments, the hydrophobe extends from the 6-position by 4-5 Å and lie above or below the plane by 0-2 Å; the position of the lone-pair of electrons extends from the 6-position by 4-5 Å and lies above or below the plane by 0-2 Å; the volume that the hydrophobe occupies is 80-100 Å³.

The hydrophobe can be linked to the pyrimidine by linker L selected from piperazine, piperidine, azetidine, pyrrolidine, octahydropyrrolo[3,4-c]pyrrole, pyrrolidine, or a C₃-C₅ alkylidene chain with up to 3 CH₂ groups being replaced with —NH—, —NHCO— or —CONH—;.

The hydrophobe can be part of a ring such as a C₃-C₅ carbocycle selected from cyclopropyl, cyclobutyl, cyclopentyl, or a phenyl ring; or a C₄-C₆ heterocycle selected from oxetane, pyrrolidine or piperidine, or a branched or unbranched C₁-C₅ alkyl chain selected from methyl, ethyl, propyl, isopropyl, butyl, sec-butyl, and tert-butyl.

The carbocycle, phenyl ring, heterocycle or alkyl chain can be optionally substituted with alkyl groups, hydroxy, alkoxy groups, and halogen atoms, preferably fluorine. The lone pair of electrons can be from a nitrogen such as a secondary or tertiary amine or a nitrile group, or an oxygen such as a alcohol, ether or carbonyl group, or a halogen such as fluorine.

One embodiment provides a pharmacophore comprising a lipophilic group and a lone pair of electrons extending from the 6-position of compounds of Table 1.

This invention also provides compounds that fit the pharmacophore. In some embodiments, said compounds are compounds of formula I:

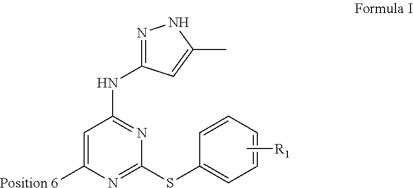

Or a pharmaceutically acceptable salt thereof, wherein

R¹ is —NHC(O)R², OR³; or two R¹ groups, taken together, form a fused phenyl ring;

R² is CH₂CH₃, CH₂CF₃, CH₂CH₂CF₃,

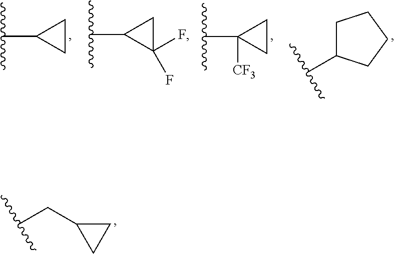

or phenyl optionally substituted with halo, CF₃, or C₁₋₃alkyl; and

R³ is C₁₋₄alkyl, C₃₋₆cycloalkyl.

In some embodiments, R¹ is —NHC(O)R²; R² is CH₂CH₃, CH₂CF₃, CH₂CH₂CF₃,

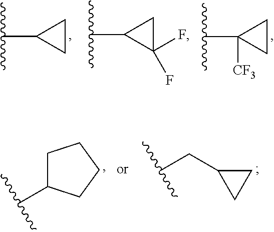

and R³ is C₁₋₄alkyl, C₃₋₆cycloalkyl.

Representative compounds that fulfill the pharmacophore and have ratio of AurB Ki/AurB Ki*>3 are shown in Table 1 below (Compounds 1-36):

TABLE 1

| | |
|---|---|
| 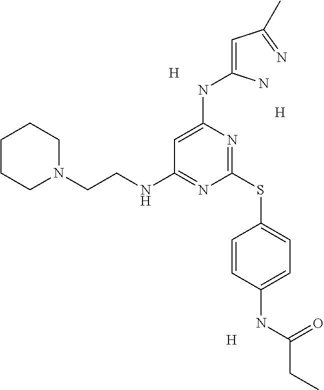 | 1 |
| 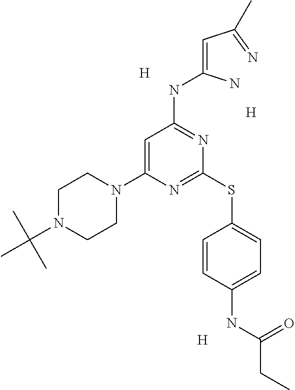 | 2 |
| 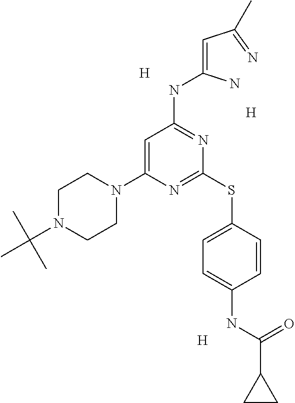 | 3 |
| 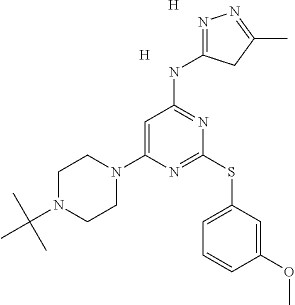 | 4 |
| 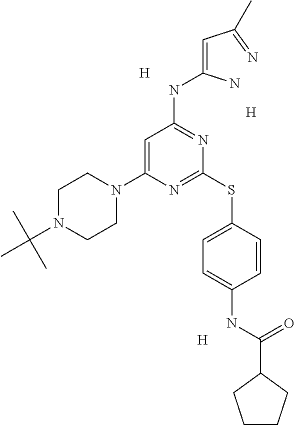 | 5 |
| 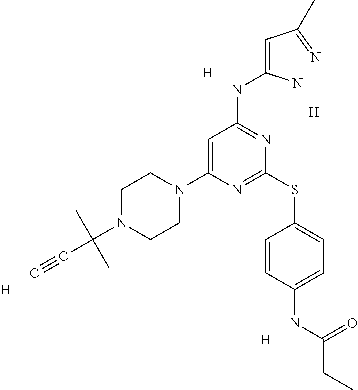 | 6 |
| 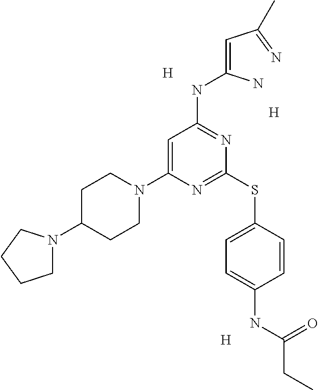 | 7 |
| 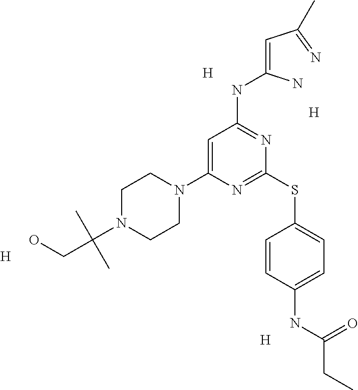 | 8 |
| 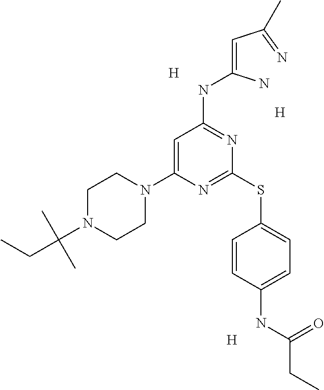 | 9 |
| 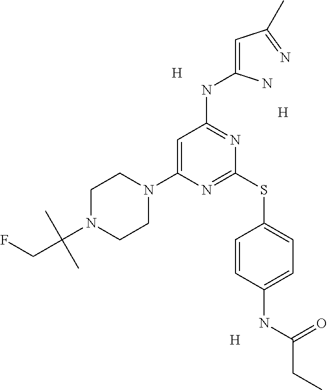 | 10 |
| 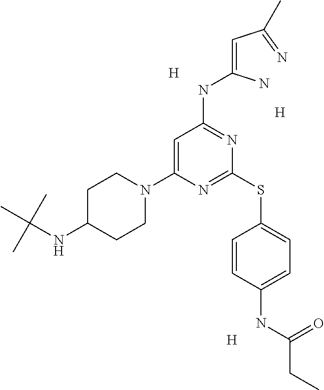 | 11 |
| 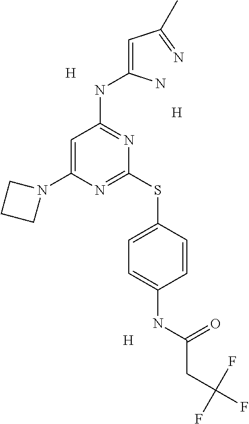 | 12 |
| 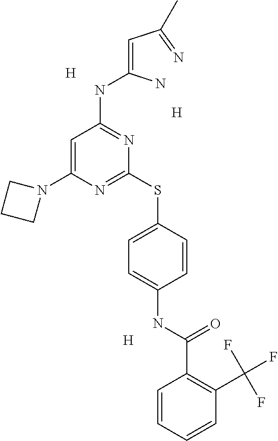 | 13 |
| 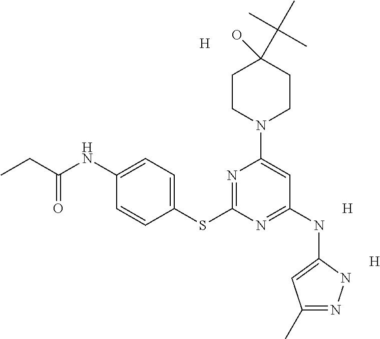 | 14 |
| 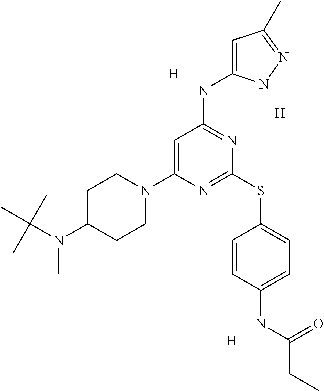 | 15 |
| 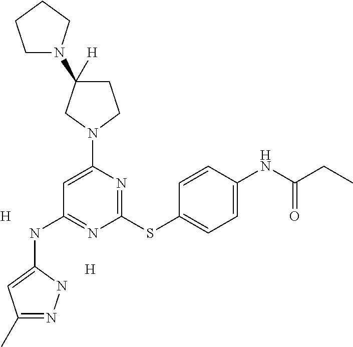 | 16 |
| 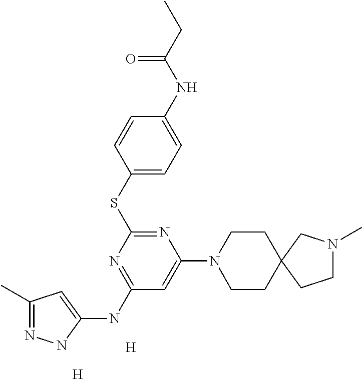 | 17 |
| 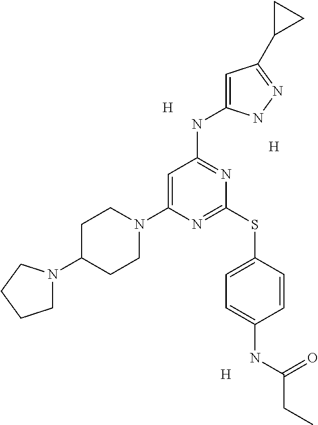 | 18 |
| 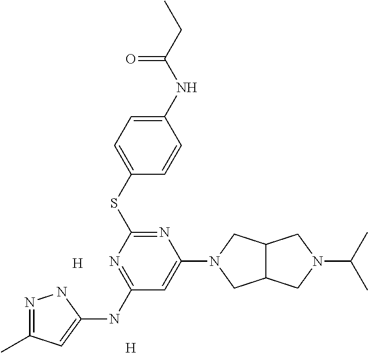 | 19 |
| 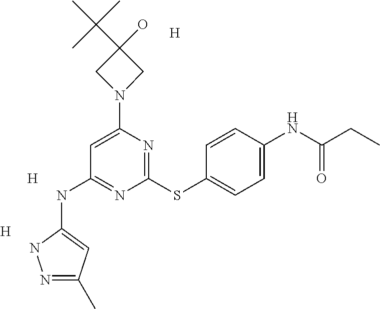 | 20 |
| 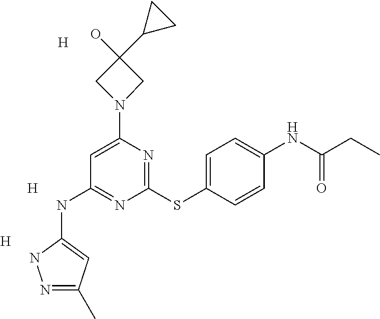 | 21 |
| 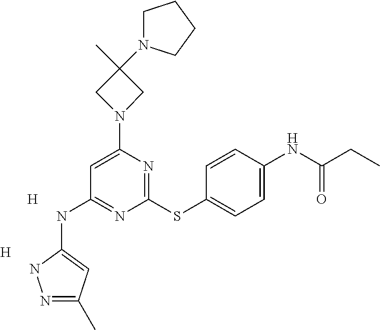 | 22 |
| 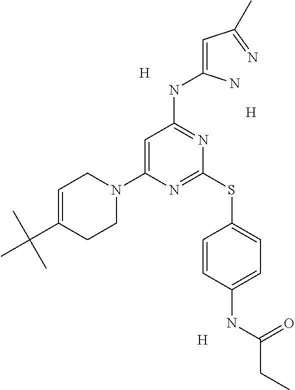 | 23 |
| 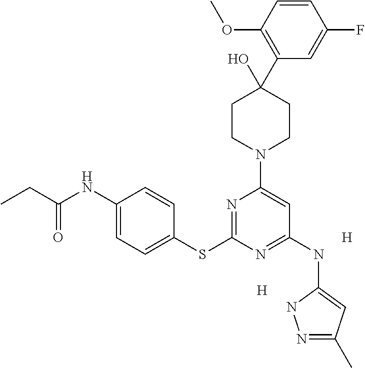 | 24 |
| 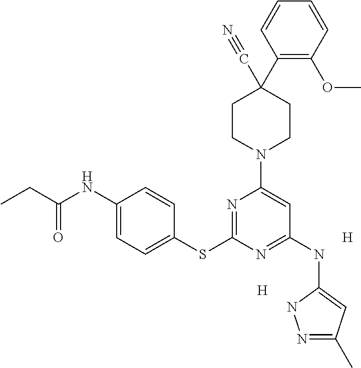 | 25 |
| 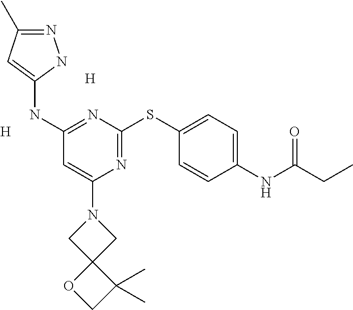 | 26 |
| 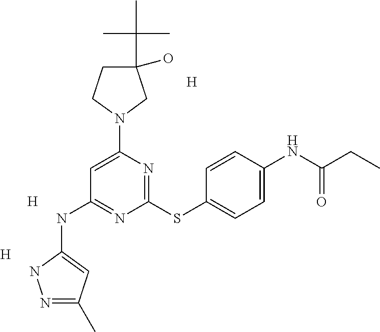 | 27 |
| 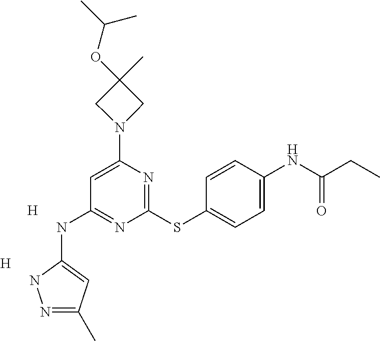 | 28 |
| 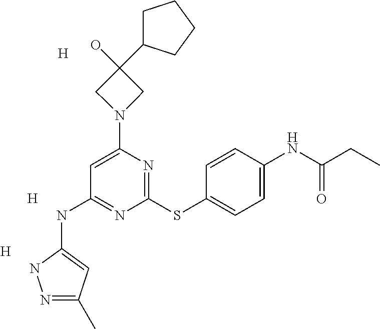 | 29 |
| 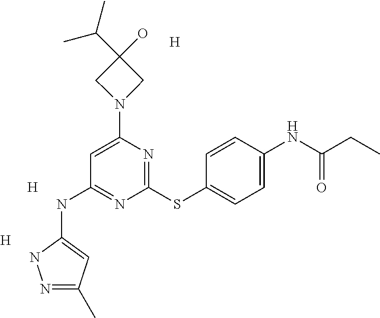 | 30 |
| 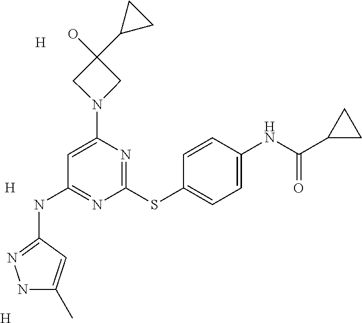 | 31 |
| 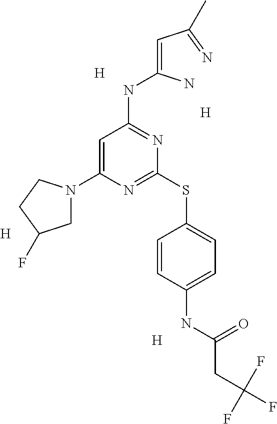 | 32 |
| 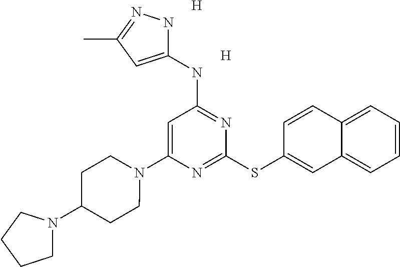 | 33 |
| 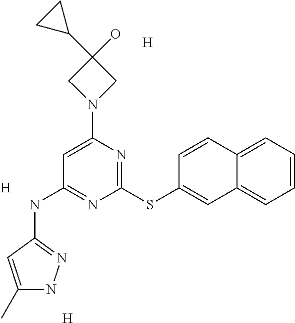 | 34 |
| 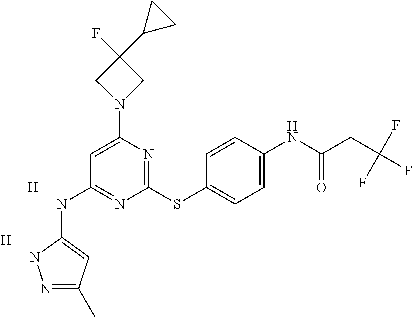 | 35 |
| 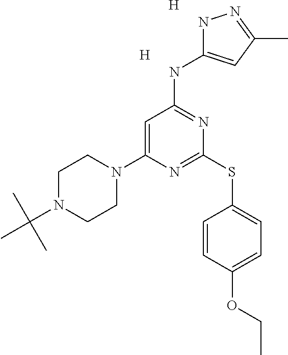 | 36 |

One embodiment provides the compounds shown in Table 1 (compounds 1-36). Another embodiment provides the following compounds: 3-6, 8-10, 23, 33, and 36. Yet another embodiment provides the following compounds: 3-6, 8-10, 23, and 36.

This invention also provides methods for identifying, evaluating, selecting, prioritizing, designing, and screening for Aurora inhibitors (in some embodiments, Aurora B inhibitors). One embodiment provides a method for selecting an Aurora B kinase inhibitor by 1) assaying according to a method of this invention; and/or 2) modeling to evaluate fit to pharmacophore. Another embodiment provides a drug discovery method for identifying Aurora B kinase inhibitors comprising 1) assaying a compound according to a method of this invention; and/or 2) modeling the compound to evaluate fit to pharmacophore; 3) selecting the compound if it meets one or both (preferably both criteria). Another embodiment provides a drug discovery method for prioritizing Aurora B kinase inhibitors for further evaluation comprising the step of selecting compounds with a Ki/Ki* ratio of >3. Some embodiments comprise the step of selecting compounds with a Ki/Ki* ratio of >1.

As would be recognized by skilled practitioners there are various ways to obtain the Ki values that are called for by this invention. In practicing this invention, such values may be determined by known methods (see Examples 4 and 5) or otherwise obtained. Ki* values are obtained according to a method of this invention.

Another embodiment provides compounds identified or selected according to the methods described herein. In some embodiments, said compounds are selected by assaying a compound according to the methods described herein. In some embodiments, the compounds have a Ki/Ki* of greater than 1. In other embodiments, the compounds have a Ki/Ki* of greater than 3. In yet other embodiments, said compounds are selected by modeling the compound to evaluate its fit to a pharmacophore described herein (based on Formula I: see paragraphs [0034] and [0035]). In other embodiments, said compounds are selected by 1) assaying a compound according to a method of this invention; and/or 2) modeling the compound to evaluate fit to the pharmacophore; and 3) selecting the compound if it meets one or both (preferably both criteria).

This invention also provides a compound having the features of the pharmacophore. In some embodiments, said compound is not one of the following compounds from Table 1: compound 1-2, 7, 11-22, 24-32, or 34-35. In other embodiments, said compound is compound 3-6, 8-10, 23, or 36.

Applicants' methods also relate to the cross-reactivity of Aurora inhibitors with other kinases. Closed conformations are not common in protein kinases. Applicants' method for using this structural and kinetic modeling may also be used in methods related to identifying compounds with certain cross-reactivities.

Finally, another embodiment provides compounds that are useful as Aurora inhibitors. One embodiment provides the following compound:

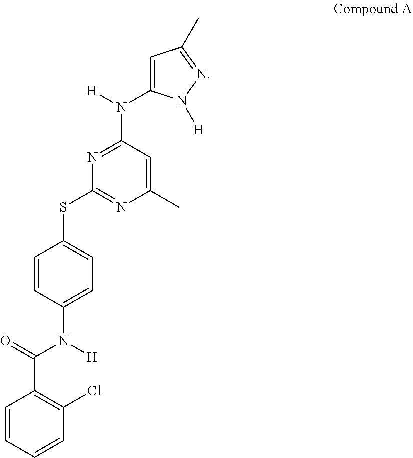 [SMILES: Cc1cc(Nc2cc(C)nc(Sc3ccc(NC(=O)c4ccccc4Cl)cc3)n2)[nH]n1]

For purposes of this invention, the chemical elements are identified in accordance with the Periodic Table of the Elements, CAS version, Handbook of Chemistry and Physics, 75^{th} Ed. Additionally, general principles of organic chemistry are described in texts known to those of ordinary skill in the art, including, for example, “Organic Chemistry”, Thomas Sorrell, University Science Books, Sausalito: 1999, and “March's Advanced Organic Chemistry”, 5^{th} Ed Ed.: Smith, M. B. and March, J., John Wiley & Sons, New York: 2001, the entire contents of which are hereby incorporated by reference.

As described herein, a specified number range of atoms includes any integer therein. For example, a group having from 1-4 atoms could have 1, 2, 3, or 4 atoms.

As described herein, compounds of the invention may optionally be substituted with one or more substituents, such as are illustrated generally above, or as exemplified by particular classes, subclasses, and species of the invention. It will be appreciated that the phrase “optionally substituted” is used interchangeably with the phrase “substituted or unsubstituted.” In general, the term “substituted”, whether preceded by the term “optionally” or not, refers to the replacement of hydrogen radicals in a given structure with the radical of a specified substituent. Unless otherwise indicated, an optionally substituted group may have a substituent at each substitutable position of the group, and when more than one position in any given structure may be substituted with more than one substituent selected from a specified group, the substituent may be either the same or different at every position. Combinations of substituents envisioned by this invention are preferably those that result in the formation of stable or chemically feasible compounds.

The term “stable”, as used herein, refers to compounds that are not substantially altered when subjected to conditions to allow for their production, detection, and preferably their recovery, purification, and use for one or more of the purposes disclosed herein. In some embodiments, a stable compound or chemically feasible compound is one that is not substantially altered when kept at a temperature of 40° C. or less, in the absence of moisture or other chemically reactive conditions, for at least a week.

The term “cycloaliphatic” (or “carbocycle” or “carbocyclyl” or “cycloalkyl” and the like) refers to a monocyclic C₃-C₈ hydrocarbon or bicyclic C₈-C₁₂ hydrocarbon that is completely saturated or that contains one or more units of unsaturation, but which is not aromatic, that has a single point of attachment to the rest of the molecule wherein any individual ring in said bicyclic ring system has 3-7 members. Suitable cycloaliphatic groups include, but are not limited to, cycloalkyl and cycloalkenyl groups. Specific examples include, but are not limited to, cyclohexyl, cyclopropenyl, and cyclobutyl.

The term “alkyl” as used herein, means an unbranched or branched, straight-chain or cyclic hydrocarbon that is completely saturated and has a single point of attachment to the rest of the molecule. Unless otherwise indicated, alkyl groups contain 1-12 carbon atoms. Specific examples of alkyl groups include, but are not limited to, methyl, ethyl, isopropyl, n-propyl, and sec-butyl.

In the compounds of this invention, rings include linearly-fused, bridged, or spirocyclic rings. Examples of bridged cycloaliphatic groups include, but are not limited to, bicyclo[3.3.2]decane, bicyclo[3.1.1]heptane, and bicyclo[3.2.2]nonane.

The term “heterocycle”, “heterocyclyl”, or “heterocyclic”, and the like, as used herein means non-aromatic, monocyclic or bicyclic ring in which one or more ring members are an independently selected heteroatom. In some embodiments, the “heterocycle”, “heterocyclyl”, or “heterocyclic” group has three to ten ring members in which one or more ring members is a heteroatom independently selected from oxygen, sulfur, nitrogen, or phosphorus, and each ring in the system contains 3 to 7 ring members. Examples of bridged heterocycles include, but are not limited to, 7-aza-bicyclo[2.2.1]heptane and 3-aza-bicyclo[3.2.2]nonane.

Suitable heterocycles include, but are not limited to, 3-1H-benzimidazol-2-one, 3-(1-alkyl)-benzimidazol-2-one, 2-tetrahydrofuranyl, 3-tetrahydrofuranyl, 2-tetrahydrothiophenyl, 3-tetrahydrothiophenyl, 2-morpholino, 3-morpholino, 4-morpholino, 2-thiomorpholino, 3-thiomorpholino, 4-thiomorpholino, 1-pyrrolidinyl, 2-pyrrolidinyl, 3-pyrrolidinyl, 1-tetrahydropiperazinyl, 2-tetrahydropiperazinyl, 3-tetrahydropiperazinyl, 1-piperidinyl, 2-piperidinyl, 3-piperidinyl, 1-pyrazolinyl, 3-pyrazolinyl, 4-pyrazolinyl, 5-pyrazolinyl, 1-piperidinyl, 2-piperidinyl, 3-piperidinyl, 4-piperidinyl, 2-thiazolidinyl, 3-thiazolidinyl, 4-thiazolidinyl, 1-imidazolidinyl, 2-imidazolidinyl, 4-imidazolidinyl, 5-imidazolidinyl, indolinyl, tetrahydroquinolinyl, tetrahydroisoquinolinyl, benzothiolane, benzodithiane, and 1,3-dihydro-imidazol-2-one.

The term “heteroatom” means one or more of oxygen, sulfur, nitrogen, phosphorus, or silicon (including, any oxidized form of nitrogen, sulfur, phosphorus, or silicon; the quaternized form of any basic nitrogen or; a substitutable nitrogen of a heterocyclic ring, for example N (as in 3,4-dihydro-2H-pyrrolyl), NH (as in pyrrolidinyl) or NR⁺ (as in N-substituted pyrrolidinyl)).

The term “aryl” refers to monocyclic, or bicyclic ring having a total of five to twelve ring members, wherein at least one ring in the system is aromatic and wherein each ring in the system contains 3 to 7 ring members. The term “aryl” may be used interchangeably with the term “aryl ring”. The term “aryl” also refers to heteroaryl ring systems as defined hereinbelow.

The term “heteroaryl”, refers to monocyclic or bicyclic ring having a total of five to twelve ring members, wherein at least one ring in the system is aromatic, at least one ring in the system contains one or more heteroatoms, and wherein each ring in the system contains 3 to 7 ring members. The term “heteroaryl” may be used interchangeably with the term “heteroaryl ring” or the term “heteroaromatic”. Suitable heteroaryl rings include, but are not limited to, 2-furanyl, 3-furanyl, N-imidazolyl, 2-imidazolyl, 4-imidazolyl, 5-imidazolyl, benzimidazolyl, 3-isoxazolyl, 4-isoxazolyl, 5-isoxazolyl, 2-oxazolyl, 4-oxazolyl, 5-oxazolyl, N-pyrrolyl, 2-pyrrolyl, 3-pyrrolyl, 2-pyridyl, 3-pyridyl, 4-pyridyl, 2-pyrimidinyl, 4-pyrimidinyl, 5-pyrimidinyl, pyridazinyl (e.g., 3-pyridazinyl), 2-thiazolyl, 4-thiazolyl, 5-thiazolyl, tetrazolyl (e.g., 5-tetrazolyl), triazolyl (e.g., 2-triazolyl and 5-triazolyl), 2-thienyl, 3-thienyl, benzofuryl, benzothiophenyl, indolyl (e.g., 2-indolyl), pyrazolyl (e.g., 2-pyrazolyl), isothiazolyl, 1,2,3-oxadiazolyl, 1,2,5-oxadiazolyl, 1,2,4-oxadiazolyl, 1,2,3-triazolyl, 1,2,3-thiadiazolyl, 1,3,4-thiadiazolyl, 1,2,5-thiadiazolyl, purinyl, pyrazinyl, 1,3,5-triazinyl, quinolinyl (e.g., 2-quinolinyl, 3-quinolinyl, 4-quinolinyl), and isoquinolinyl (e.g., 1-isoquinolinyl, 3-isoquinolinyl, or 4-isoquinolinyl).

The term “unsaturated”, as used herein, means that a moiety has one or more units of unsaturation.

The term “halogen” means F, Cl, Br, or I.

Unless otherwise indicated, structures depicted herein are also meant to include all isomeric (e.g., enantiomeric, diastereomeric, and geometric (or conformational)) forms of the structure; for example, the R and S configurations for each asymmetric center, (Z) and (E) double bond isomers, and (Z) and (E) conformational isomers. Therefore, single stereochemical isomers as well as enantiomeric, diastereomeric, and geometric (or conformational) mixtures of the present compounds are within the scope of the invention.

Unless otherwise indicated, all tautomeric forms of the compounds of the invention are within the scope of the invention. As would be understood by a skilled practitioner, a pyrazole group can be represented in a variety of ways. For example, a structure drawn as

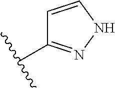 [SMILES: *c1cc[nH]n1]

represents other possible tautomers, such as

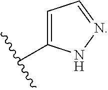 [SMILES: *c1ccn[nH]1]

Likewise, a structure drawn as

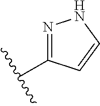 [SMILES: *c1cc[nH]n1]

also represents other possible tautomers, such as

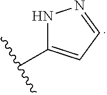 [SMILES: *c1ccn[nH]1]

Unless otherwise indicated, a substituent can freely rotate around any rotatable bonds. For example, a substituent drawn as

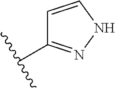 [SMILES: *c1cc[nH]n1]

also represents

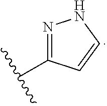 [SMILES: *c1cc[nH]n1]

Likewise, a substituent drawn as

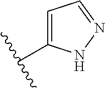 [SMILES: *c1ccn[nH]1]

also represents

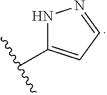 [SMILES: *c1ccn[nH]1]

Additionally, unless otherwise indicated, structures depicted herein are also meant to include compounds that differ only in the presence of one or more isotopically enriched atoms. For example, compounds having the present structures except for the replacement of hydrogen by deuterium or tritium, or the replacement of a carbon by a ¹³C- or ¹⁴C-enriched carbon are within the scope of this invention. Such compounds are useful, for example, as analytical tools or probes in biological assays.

### Synthesis

The compounds of this invention may be prepared according to the General Scheme show below:

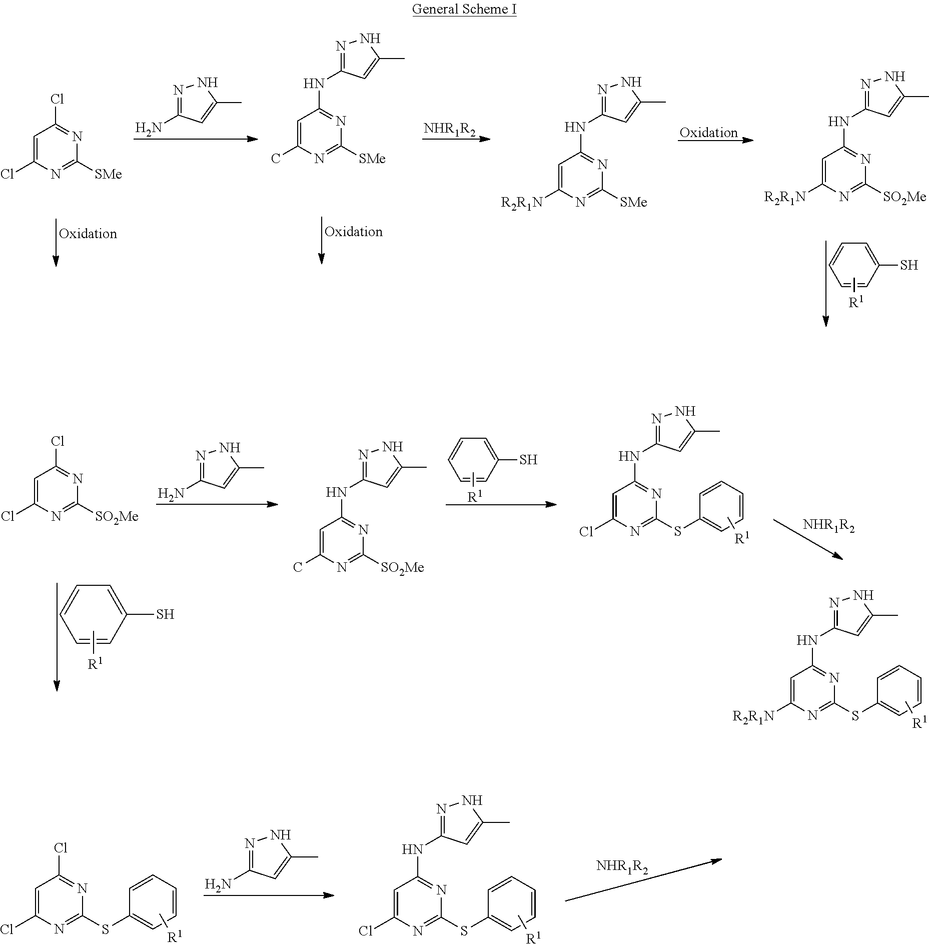

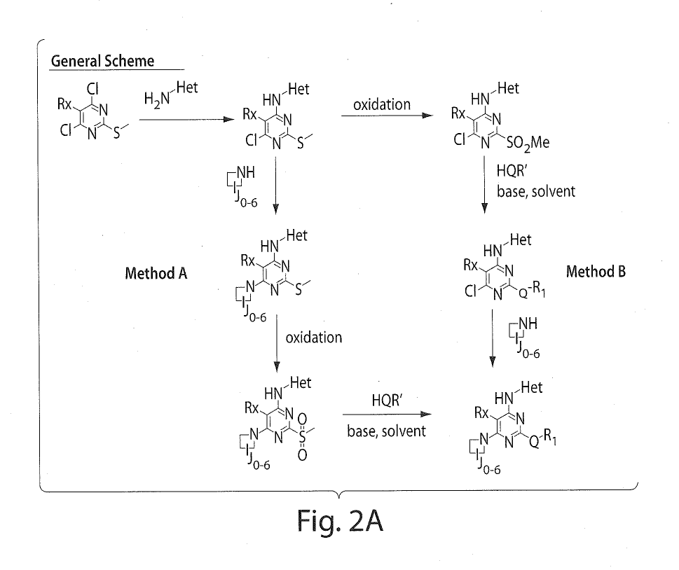

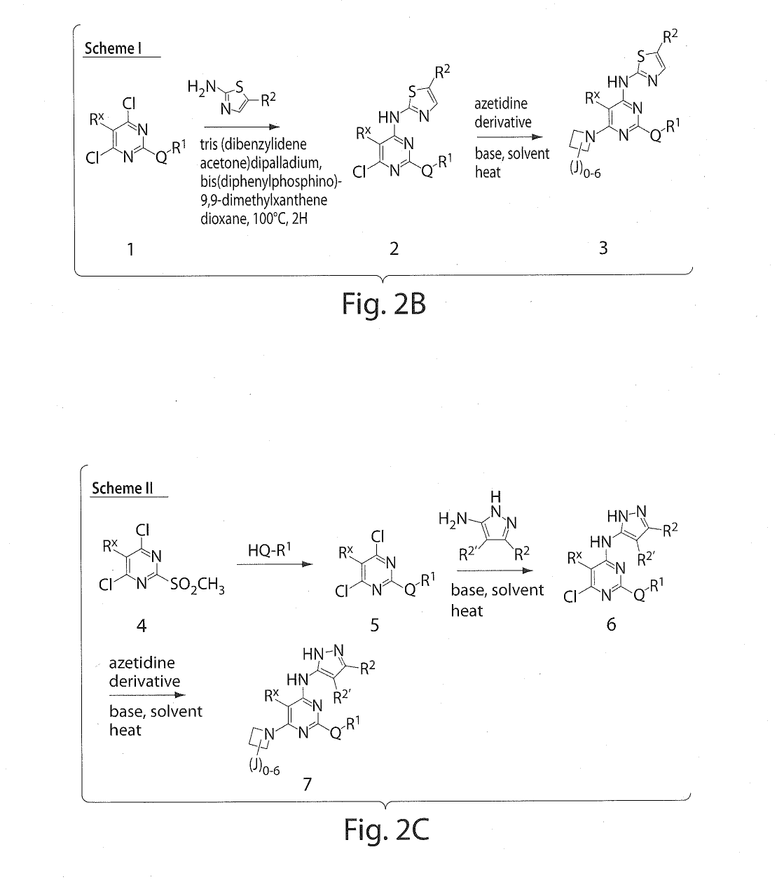

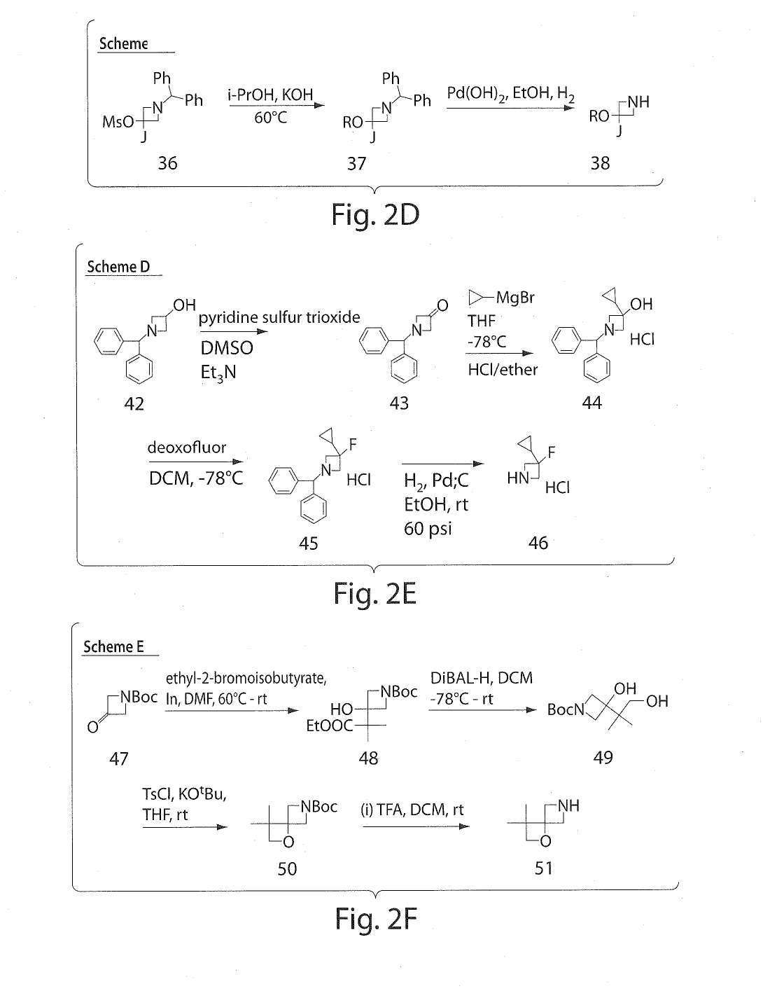

Scheme I above shows a generic method for making compounds of this invention. The compounds of this invention can be made in a variety of ways, as shown above. In essence, there are three main groups that are added to the dichloropyrimidine starting material. The order in which these groups are added can vary. The three main reactions involved are: addition of the amine (NHR₁R₂); addition of the aminopyrazole, and addition of Ph-SH (which includes the oxidation of —SMe into a suitable leaving group, e.g., SO₂Me). As shown above, these three groups can be added in various different orders. For instance, the aminopyrazole can be added first, followed by addition of NHR₁R₂, oxidation, and finally addition of Ph-SH. Or instead, oxidation can occur first, followed by addition of Ph-SH, addition of the aminopyrazole, and finally addition of NHR₁R₂. A skilled practitioner would understand the various reactions shown above. Additional schemes and experimentals are described herein and also in FIG. 2.

In some embodiments, the benzenethiol (Ph-SH) displaces the SO₂Me leaving group under heating conditions in the presence of a suitable solvent (e.g. t-BuOH) for 16 hours. In other embodiments, displacement of the SO₂Me leaving group is done at 0° C. in the presence of acetonitrile and triethylamine for 1 hour. In some embodiments, addition of the aminopyrazole is done by heating the amino-pyrazole and the chloropyrimidine intermediate in the presence of a suitable solvent (e.g. DMF) and a suitable base (e.g. DIPEA/NaI). In some embodiments, addition of the amine (NR₁R₂) occurs by heating the amine (NR₁R₂) and the chloropyrimidine intermediate in the presence of a suitable solvent (e.g. n-BuOH).

The compounds may also be prepared using steps generally known to those of ordinary skill in the art (see e.g., WO2002/057259, WO2004/000833, WO 2007/056221, WO 2007/056163, and WO 2007/056164, the entire contents of which are hereby incorporated by reference) and/or according to the Schemes and Examples herein.

Those compounds may be analyzed by known methods, including but not limited to LCMS (liquid chromatography mass spectrometry) and NMR (nuclear magnetic resonance). It should be understood that the specific conditions shown below are only examples, and are not meant to limit the scope of the conditions that can be used for making compounds of this invention. Instead, this invention also includes conditions that would be apparent to those skilled in that art in light of this specification for making the compounds of this invention.

Methods for evaluating the activity of the compounds of this invention (e.g., kinase assays) are known in the art and are also described in the examples set forth.

The activity of the compounds as protein kinase inhibitors may be assayed in vitro, in vivo or in a cell line. In vitro assays include assays that determine inhibition of either the kinase activity or ATPase activity of the activated kinase. Alternate in vitro assays quantitate the ability of the inhibitor to bind to the protein kinase and may be measured either by radiolabelling the inhibitor prior to binding, isolating the inhibitor/kinase complex and determining the amount of radiolabel bound, or by running a competition experiment where new inhibitors are incubated with the kinase bound to known radioligands.

Another aspect of the invention relates to inhibiting kinase activity in a biological sample, which method comprises contacting said biological sample with a compound of formula I or a composition comprising said compound. The term “biological sample”, as used herein, means an in vitro or an ex vivo sample, including, without limitation, cell cultures or extracts thereof; biopsied material obtained from a mammal or extracts thereof; and blood, saliva, urine, feces, semen, tears, or other body fluids or extracts thereof

Inhibition of kinase activity in a biological sample is useful for a variety of purposes that are known to one of skill in the art. Examples of such purposes include, but are not limited to, blood transfusion, organ-transplantation, biological specimen storage, and biological assays.

Inhibition of kinase activity in a biological sample is also useful for the study of kinases in biological and pathological phenomena; the study of intracellular signal transduction pathways mediated by such kinases; and the comparative evaluation of new kinase inhibitors.

The Aurora protein kinase inhibitors or pharmaceutical salts thereof may be formulated into pharmaceutical compositions for administration to animals or humans. These pharmaceutical compositions, which comprise an amount of the Aurora protein inhibitor effective to treat or prevent an Aurora-mediated condition and a pharmaceutically acceptable carrier, are another embodiment of the present invention.

The term “Aurora-mediated condition” or “Aurora-mediated disease” as used herein means any disease or other deleterious condition in which Aurora (Aurora A, Aurora B, and Aurora C) is known to play a role. Such conditions include, without limitation, cancer, proliferative disorders, and myeloproliferative disorders.

Examples of myeloproliferative disorders include, but are not limited, to, polycythemia vera, thrombocythemia, myeloid metaplasia with myelofibrosis, chronic myelogenous leukaemia (CML), chronic myelomonocytic leukemia, hypereosinophilic syndrome, juvenile myelomonocytic leukemia, and systemic mast cell disease.

The term “cancer” also includes, but is not limited to, the following cancers: epidermoid Oral: buccal cavity, lip, tongue, mouth, pharynx; Cardiac: sarcoma (angiosarcoma, fibrosarcoma, rhabdomyosarcoma, liposarcoma), myxoma, rhabdomyoma, fibroma, lipoma and teratoma; Lung: bronchogenic carcinoma (squamous cell or epidermoid, undifferentiated small cell, undifferentiated large cell, adenocarcinoma), alveolar (bronchiolar) carcinoma, bronchial adenoma, sarcoma, lymphoma, chondromatous hamartoma, mesothelioma; Gastrointestinal: esophagus (squamous cell carcinoma, larynx, adenocarcinoma, leiomyosarcoma, lymphoma), stomach (carcinoma, lymphoma, leiomyosarcoma), pancreas (ductal adenocarcinoma, insulinoma, glucagonoma, gastrinoma, carcinoid tumors, vipoma), small bowel or small intestines (adenocarcinoma, lymphoma, carcinoid tumors, Karposi's sarcoma, leiomyoma, hemangioma, lipoma, neurofibroma, fibroma), large bowel or large intestines (adenocarcinoma, tubular adenoma, villous adenoma, hamartoma, leiomyoma), colon, colon-rectum, colorectal; rectum, Genitourinary tract: kidney (adenocarcinoma, Wilm's tumor [nephroblastoma], lymphoma, leukemia), bladder and urethra (squamous cell carcinoma, transitional cell carcinoma, adenocarcinoma), prostate (adenocarcinoma, sarcoma), testis (seminoma, teratoma, embryonal carcinoma, teratocarcinoma, choriocarcinoma, sarcoma, interstitial cell carcinoma, fibroma, fibroadenoma, adenomatoid tumors, lipoma); Liver: hepatoma (hepatocellular carcinoma), cholangiocarcinoma, hepatoblastoma, angiosarcoma, hepatocellular adenoma, hemangioma, biliary passages; Bone: osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochronfroma (osteocartilaginous exostoses), benign chondroma, chondroblastoma, chondromyxofibroma, osteoid osteoma and giant cell tumors; Nervous system: skull (osteoma, hemangioma, granuloma, xanthoma, osteitis deformans), meninges (meningioma, meningiosarcoma, gliomatosis), brain (astrocytoma, medulloblastoma, glioma, ependymoma, germinoma [pinealoma], glioblastoma multiform, oligodendroglioma, schwannoma, retinoblastoma, congenital tumors), spinal cord neurofibroma, meningioma, glioma, sarcoma); Gynecological: uterus (endometrial carcinoma), cervix (cervical carcinoma, pre-tumor cervical dysplasia), ovaries (ovarian carcinoma [serous cystadenocarcinoma, mucinous cystadenocarcinoma, unclassified carcinoma], granulosa-thecal cell tumors, Sertoli-Leydig cell tumors, dysgerminoma, malignant teratoma), vulva (squamous cell carcinoma, intraepithelial carcinoma, adenocarcinoma, fibrosarcoma, melanoma), vagina (clear cell carcinoma, squamous cell carcinoma, botryoid sarcoma (embryonal rhabdomyosarcoma), fallopian tubes (carcinoma), breast; Hematologic: blood (myeloid leukemia [acute and chronic], acute lymphoblastic leukemia, chronic lymphocytic leukemia, myeloproliferative diseases, multiple myeloma, myelodysplastic syndrome), Hodgkin's disease, non-Hodgkin's lymphoma [malignant lymphoma] hairy cell; lymphoid disorders; Skin: malignant melanoma, basal cell carcinoma, squamous cell carcinoma, Karposi's sarcoma, keratoacanthoma, moles dysplastic nevi, lipoma, angioma, dermatofibroma, keloids, psoriasis, Thyroid gland: papillary thyroid carcinoma, follicular thyroid carcinoma; medullary thyroid carcinoma, undifferentiated thyroid cancer, multiple endocrine neoplasia type 2A, multiple endocrine neoplasia type 2B, familial medullary thyroid cancer, pheochromocytoma, paraganglioma; and Adrenal glands: neuroblastoma. Thus, the term “cancerous cell” as provided herein, includes a cell afflicted by any one of the above-identified conditions. In some embodiments, the cancer is selected from colorectal, thyroid, or breast cancer.

In some embodiments, the compounds of this invention are useful for treating cancer, such as colorectal, thyroid, breast, and lung cancer; and myeloproliferative disorders, such as polycythemia vera, thrombocythemia, myeloid metaplasia with myelofibrosis, chronic myelogenous leukemia, chronic myelomonocytic leukemia, hypereosinophilic syndrome, juvenile myelomonocytic leukemia, and systemic mast cell disease.

In some embodiments, the compounds of this invention are useful for treating hematopoietic disorders, in particular, acute-myelogenous leukemia (AML), chronic-myelogenous leukemia (CML), acute-promyelocytic leukemia (APL), and acute lymphocytic leukemia (ALL).

In addition to the compounds of this invention, pharmaceutically acceptable derivatives or prodrugs of the compounds of this invention may also be employed in compositions to treat or prevent the above-identified disorders.

A “pharmaceutically acceptable derivative or prodrug” means any pharmaceutically acceptable ester, salt of an ester or other derivative of a compound of this invention which, upon administration to a recipient, is capable of providing, either directly or indirectly, a compound of this invention or an inhibitorily active metabolite or residue thereof. Such derivatives or prodrugs include those that increase the bioavailability of the compounds of this invention when such compounds are administered to a patient (e.g., by allowing an orally administered compound to be more readily absorbed into the blood) or which enhance delivery of the parent compound to a biological compartment (e.g., the brain or lymphatic system) relative to the parent species.

Examples of pharmaceutically acceptable prodrugs of the compounds of this invention include, without limitation, esters, amino acid esters, phosphate esters, metal salts and sulfonate esters.

The compounds of this invention can exist in free form for treatment, or where appropriate, as a pharmaceutically acceptable salt.

As used herein, the term “pharmaceutically acceptable salt” refers to salts of a compound which are, within the scope of sound medical judgment, suitable for use in contact with the tissues of humans and lower animals without undue toxicity, irritation, allergic response and the like, and are commensurate with a reasonable benefit/risk ratio.

Pharmaceutically acceptable salts of the compounds of this invention include those derived from suitable inorganic and organic acids and bases. These salts can be prepared in situ during the final isolation and purification of the compounds. Acid addition salts can be prepared by 1) reacting the purified compound in its free-based form with a suitable organic or inorganic acid and 2) isolating the salt thus formed.

Examples of suitable acid salts include acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, formate, fumarate, glucoheptanoate, glycerophosphate, glycolate, hemisulfate, heptanoate, hexanoate, hydrochloride, hydrobromide, hydroiodide, 2-hydroxyethanesulfonate, lactate, maleate, malonate, methanesulfonate, 2-naphthalenesulfonate, nicotinate, nitrate, oxalate, palmoate, pectinate, persulfate, 3-phenylpropionate, phosphate, picrate, pivalate, propionate, salicylate, succinate, sulfate, tartrate, thiocyanate, tosylate and undecanoate. Other acids, such as oxalic, while not in themselves pharmaceutically acceptable, may be employed in the preparation of salts useful as intermediates in obtaining the compounds of the invention and their pharmaceutically acceptable acid addition salts.

Base addition salts can be prepared by 1) reacting the purified compound in its acid form with a suitable organic or inorganic base and 2) isolating the salt thus formed.

Salts derived from appropriate bases include alkali metal (e.g., sodium and potassium), alkaline earth metal (e.g., magnesium), ammonium and N⁺(C₁₋₄ alkyl)₄ salts. This invention also envisions the quaternization of any basic nitrogen-containing groups of the compounds disclosed herein. Water or oil-soluble or dispersible products may be obtained by such quaternization.

Base addition salts also include alkali or alkaline earth metal salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, loweralkyl sulfonate and aryl sulfonate. Other acids and bases, while not in themselves pharmaceutically acceptable, may be employed in the preparation of salts useful as intermediates in obtaining the compounds of the invention and their pharmaceutically acceptable acid or base addition salts.

Pharmaceutically acceptable carriers that may be used in these pharmaceutical compositions include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

The compositions of the present invention may be administered orally, parenterally, by inhalation spray, topically, rectally, nasally, buccally, vaginally or via an implanted reservoir. The term “parenteral” as used herein includes subcutaneous, intravenous, intramuscular, intra-articular, intra-synovial, intrasternal, intrathecal, intraperitoneal, intrahepatic, intralesional and intracranial injection or infusion techniques.

Sterile injectable forms of the compositions of this invention may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally-acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose, a bland fixed oil may be employed including synthetic mono- or di-glycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as carboxymethyl cellulose or similar dispersing agents which are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation.

The pharmaceutical compositions of this invention may be orally administered in any orally acceptable dosage form including, but not limited to, capsules, tablets, aqueous suspensions or solutions. In the case of tablets for oral use, carriers commonly used may include lactose and corn starch. Lubricating agents, such as magnesium stearate, may also be added. For oral administration in a capsule form, useful diluents may include lactose and dried cornstarch. When aqueous suspensions are required for oral use, the active ingredient may be combined with emulsifying and suspending agents. If desired, certain sweetening, flavoring or coloring agents may also be added.

Alternatively, the pharmaceutical compositions of this invention may be administered in the form of suppositories for rectal administration. These can be prepared by mixing the agent with a suitable non-irritating excipient which is solid at room temperature but liquid at rectal temperature and therefore will melt in the rectum to release the drug. Such materials may include cocoa butter, beeswax and polyethylene glycols.

The pharmaceutical compositions of this invention may also be administered topically, especially when the target of treatment includes areas or organs readily accessible by topical application, including diseases of the eye, the skin, or the lower intestinal tract. Suitable topical formulations may be prepared for each of these areas or organs.

Topical application for the lower intestinal tract can be effected in a rectal suppository formulation (see above) or in a suitable enema formulation. Topically-transdermal patches may also be used.

For topical applications, the pharmaceutical compositions may be formulated in a suitable ointment containing the active component suspended or dissolved in one or more carriers. Carriers for topical administration of the compounds of this invention may include, but are not limited to, mineral oil, liquid petrolatum, white petrolatum, propylene glycol, polyoxyethylene, polyoxypropylene compound, emulsifying wax and water. Alternatively, the pharmaceutical compositions may be formulated in a suitable lotion or cream containing the active components suspended or dissolved in one or more pharmaceutically acceptable carriers. Suitable carriers may include, but are not limited to, mineral oil, sorbitan monostearate, polysorbate 60, cetyl esters wax, cetearyl alcohol, 2-octyldodecanol, benzyl alcohol and water.

For ophthalmic use, the pharmaceutical compositions may be formulated as micronized suspensions in isotonic, pH adjusted sterile saline, or as solutions in isotonic, pH adjusted sterile saline, either with or without a preservative such as benzylalkonium chloride. Alternatively, for ophthalmic uses, the pharmaceutical compositions may be formulated in an ointment such as petrolatum.

The pharmaceutical compositions of this invention may also be administered by nasal aerosol or inhalation. Such compositions may be prepared as solutions in saline, employing benzyl alcohol or other suitable preservatives, absorption promoters to enhance bioavailability, fluorocarbons, and/or other conventional solubilizing or dispersing agents.

The amount of kinase inhibitor that may be combined with the carrier materials to produce a single dosage form will vary depending upon the host treated, the particular mode of administration, and the indication. In an embodiment, the compositions should be formulated so that a dosage of between 0.01-100 mg/kg body weight/day of the inhibitor can be administered to a patient receiving these compositions. In another embodiment, the compositions should be formulated so that a dosage of between 0.1-100 mg/kg body weight/day of the inhibitor can be administered to a patient receiving these compositions.

It should also be understood that a specific dosage and treatment regimen for any particular patient will depend upon a variety of factors, including the activity of the specific compound employed, the age, body weight, general health, sex, diet, time of administration, rate of excretion, drug combination, and the judgment of the treating physician and the severity of the particular disease being treated. The amount of inhibitor will also depend upon the particular compound in the composition.

According to another embodiment, the invention provides methods for treating or preventing cancer, a proliferative disorder, or a myeloproliferative disorder comprising the step of administering to a patient one of the herein-described compounds or pharmaceutical compositions.

The term “patient”, as used herein, means an animal, including a human.

In some embodiments, said method is used to treat or prevent a hematopoietic disorder, such as acute-myelogenous leukemia (AML), acute-promyelocytic leukemia (APL), chronic-myelogenous leukemia (CML), or acute lymphocytic leukemia (ALL).

In other embodiments, said method is used to treat or prevent myeloproliferative disorders, such as polycythemia vera, thrombocythemia, myeloid metaplasia with myelofibrosis, chronic myelogenous leukaemia (CML), chronic myelomonocytic leukemia, hypereosinophilic syndrome, juvenile myelomonocytic leukemia, and systemic mast cell disease.

In yet other embodiments, said method is used to treat or prevent cancer, such as cancers of the breast, colon, prostate, skin, pancreas, brain, genitourinary tract, lymphatic system, stomach, larynx and lung, including lung adenocarcinoma, small cell lung cancer, and non-small cell lung cancer.

Another embodiment provides a method of treating or preventing cancer comprising the step of administering to a patient a compound of formula I or a composition comprising said compound.

Another aspect of the invention relates to inhibiting kinase activity in a patient, which method comprises administering to the patient a compound of formula I or a composition comprising said compound. In some embodiments, said kinase is an Aurora kinase (Aurora A, Aurora B, Aurora C), Abl, Abl(T315I), Arg, FLT-3, JAK-2, MLK1, PLK4, Tie2, or TrkA.

Depending upon the particular conditions to be treated or prevented, additional drugs may be administered together with the compounds of this invention. In some cases, these additional drugs are normally administered to treat or prevent the same condition. For example, chemotherapeutic agents or other anti-proliferative agents may be combined with the compounds of this invention to treat proliferative diseases.

Another aspect of this invention is directed towards a method of treating cancer in a subject in need thereof, comprising the sequential or co-administration of a compound of this invention or a pharmaceutically acceptable salt thereof, and another therapeutic agent. In some embodiments, said additional therapeutic agent is selected from an anti-cancer agent, an anti-proliferative agent, or a chemotherapeutic agent.

In some embodiments, said additional therapeutic agent is selected from camptothecin, the MEK inhibitor: U0126, a KSP (kinesin spindle protein) inhibitor, adriamycin, interferons, and platinum derivatives, such as Cisplatin.

In other embodiments, said additional therapeutic agent is selected from taxanes; inhibitors of bcr-abl (such as Gleevec, dasatinib, and nilotinib); inhibitors of EGFR (such as Tarceva and Iressa); DNA damaging agents (such as cisplatin, oxaliplatin, carboplatin, topoisomerase inhibitors, and anthracyclines); and antimetabolites (such as AraC and 5-FU).

In yet other embodiments, said additional therapeutic agent is selected from camptothecin, doxorubicin, idarubicin, Cisplatin, taxol, taxotere, vincristine, tarceva, the MEK inhibitor, U0126, a KSP inhibitor, vorinostat, Gleevec, dasatinib, and nilotinib.

In another embodiment, said additional therapeutic agent is selected from Her-2 inhibitors (such as Herceptin); HDAC inhibitors (such as vorinostat), VEGFR inhibitors (such as Avastin), c-KIT and FLT-3 inhibitors (such as sunitinib), BRAF inhibitors (such as Bayer's BAY 43-9006) MEK inhibitors (such as Pfizer's PD0325901); and spindle poisons (such as Epothilones and paclitaxel protein-bound particles (such as Abraxane®).

Other therapies or anticancer agents that may be used in combination with the inventive anticancer agents of the present invention include surgery, radiotherapy (in but a few examples, gamma-radiation, neutron beam radiotherapy, electron beam radiotherapy, proton therapy, brachytherapy, and systemic radioactive isotopes, to name a few), endocrine therapy, biologic response modifiers (interferons, interleukins, and tumor necrosis factor (TNF) to name a few), hyperthermia and cryotherapy, agents to attenuate any adverse effects (e.g., antiemetics), and other approved chemotherapeutic drugs, including, but not limited to, alkylating drugs (mechlorethamine, chlorambucil, Cyclophosphamide, Melphalan, Ifosfamide), antimetabolites (Methotrexate), purine antagonists and pyrimidine antagonists (6-Mercaptopurine, 5-Fluorouracil, Cytarabile, Gemcitabine), spindle poisons (Vinblastine, Vincristine, Vinorelbine, Paclitaxel), podophyllotoxins (Etoposide, Irinotecan, Topotecan), antibiotics (Doxorubicin, Bleomycin, Mitomycin), nitrosoureas (Carmustine, Lomustine), inorganic ions (Cisplatin, Carboplatin), enzymes (Asparaginase), and hormones (Tamoxifen, Leuprolide, Flutamide, and Megestrol), Gleevec™, dexamethasone, and cyclophosphamide.

A compound of the instant invention may also be useful for treating cancer in combination with the following therapeutic agents: abarelix (Plenaxis Depot®; aldesleukin (Prokine®); Aldesleukin (Proleukin®); Alemtuzumabb (Campath®); alitretinoin (Panretin®); allopurinol (Zyloprim®); altretamine (Hexalen®); amifostine (Ethyol®); anastrozole (Arimidex®); arsenic trioxide (Trisenox®); asparaginase (Elspar®); azacitidine (Vidaza®); bevacuzimab (Avastin®); bexarotene capsules (Targretin®); bexarotene gel (Targretin®); bleomycin (Blenoxane®); bortezomib (Velcade®); busulfan intravenous (Busulfex®); busulfan oral (Myleran®); calusterone (Methosarb®); capecitabine (Xeloda®); carboplatin (Paraplatin®); carmustine (BCNU®, BiCNU®); carmustine (Gliadel®); carmustine with Polifeprosan 20 Implant (Gliadel Wafer®); celecoxib (Celebrex®); cetuximab (Erbitux®); chlorambucil (Leukeran®); cisplatin (Platinol®); cladribine (Leustatin®, 2-CdA); clofarabine (Clolar®); cyclophosphamide (Cytoxan®, Neosar®); cyclophosphamide (Cytoxan Injection®); cyclophosphamide (Cytoxan Tablet®); cytarabine (Cytosar-U®); cytarabine liposomal (DepoCyt®); dacarbazine (DTIC-Dome®); dactinomycin, actinomycin D (Cosmegen®); Darbepoetin alfa (Aranesp®); daunorubicin liposomal (DanuoXome®); daunorubicin, daunomycin (Daunorubicin®); daunorubicin, daunomycin (Cerubidine®); Denileukin diftitox (Ontak®); dexrazoxane (Zinecard®); docetaxel (Taxotere®); doxorubicin (Adriamycin PFS®); doxorubicin (Adriamycin®, Rubex®); doxorubicin (Adriamycin PFS Injection®); doxorubicin liposomal (Doxil®); dromostanolone propionate (Dromostanolone®); dromostanolone propionate (masterone Injection®); Elliott's B Solution (Elliott's B Solution®); epirubicin (Ellence®); Epoetin alfa (Epogen®); erlotinib (Tarceva®); estramustine (Emcyt®); etoposide phosphate (Etopophos®); etoposide, VP-16 (Vepesid®); exemestane (Aromasin®); Filgrastim (Neupogen®); floxuridine (intraarterial) (FUDR®); fludarabine (Fludara®); fluorouracil, 5-FU (Adrucil®); fulvestrant (Faslodex®); gefitinib (Iressa®); gemcitabine (Gemzar®); gemtuzumab ozogamicin (Mylotarg®); goserelin acetate (Zoladex Implant®); goserelin acetate (Zoladex®); histrelin acetate (Histrelin Implant®); hydroxyurea (Hydrea®); Ibritumomab Tiuxetan (Zevalin®); idarubicin (Idamycin®); ifosfamide (IFEX®); imatinib mesylate (Gleevec®); interferon alfa 2a (Roferon A®); Interferon alfa-2b (Intron A®); irinotecan (Camptosar®); lenalidomide (Revlimid®); letrozole (Femara®); leucovorin (Wellcovorin®, Leucovorin®); Leuprolide Acetate (Eligard®); levamisole (Ergamisol®); lomustine, CCNU (CeeBU®); meclorethamine, nitrogen mustard (Mustargen®); megestrol acetate (Megace®); melphalan, L-PAM (Alkeran®); mercaptopurine, 6-MP (Purinethol®); mesna (Mesnex®); mesna (Mesnex Tabs®); methotrexate (Methotrexate®); methoxsalen (Uvadex®); mitomycin C (Mutamycin®); mitotane (Lysodren®); mitoxantrone (Novantrone®); nandrolone phenpropionate (Durabolin-50®); nelarabine (Arranon®); Nofetumomab (Verluma®); Oprelvekin (Neumega®); oxaliplatin (Eloxatin®); paclitaxel (Paxene®); paclitaxel (Taxol®); paclitaxel protein-bound particles (Abraxane®); palifermin (Kepivance®); pamidronate (Aredia®); pegademase (Adagen (Pegademase Bovine)®); pegaspargase (Oncaspar®); Pegfilgrastim (Neulasta®); pemetrexed disodium (Alimta®); pentostatin (Nipent®); pipobroman (Vercyte®); plicamycin, mithramycin (Mithracin®); porfimer sodium (Photofrin®); procarbazine (Matulane®); quinacrine (Atabrine®); Rasburicase (Elitek®); Rituximab (Rituxan®); sargramostim (Leukine®); Sargramostim (Prokine®); sorafenib (Nexavar®); streptozocin (Zanosar®); sunitinib maleate (Sutent®); talc (Sclerosol®); tamoxifen (Nolvadex®); temozolomide (Temodar®); teniposide, VM-26 (Vumon®); testolactone (Teslac®); thioguanine, 6-TG (Thioguanine®); thiotepa (Thioplex®); topotecan (Hycamtin®); toremifene (Fareston R); Tositumomab (Bexxar®); Tositumomab/I-131 tositumomab (Bexxar®); Trastuzumab (Herceptin®); tretinoin, ATRA (Vesanoid®); Uracil Mustard (Uracil Mustard Capsules®); valrubicin (Valstar®); vinblastine (Velban®); vincristine (Oncovin®); vinorelbine (Navelbine®); zoledronate (Zometa®) and vorinostat (Zolinza®).

For a comprehensive discussion of updated cancer therapies see, http://www.nci.nih.gov/, a list of the FDA approved oncology drugs at http://www.fda.gov/cder/cancer/druglistframe.htm, and The Merck Manual, Seventeenth Ed. 1999, the entire contents of which are hereby incorporated by reference.

Another embodiment provides a simultaneous, separate or sequential use of a combined preparation.

Those additional agents may be administered separately, as part of a multiple dosage regimen, from the kinase inhibitor-containing compound or composition. Alternatively, those agents may be part of a single dosage form, mixed together with the kinase inhibitor in a single composition.

### EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

As used herein, the term “Rt(min)” refers to the HPLC or LCMS retention time, in minutes, associated with the compound.

Unless otherwise indicated, the HPLC method utilized to obtain the reported retention time is as follows:

Column: ACE C8 column, 4.6×150 mm

Gradient: 0-100% acetonitrile+methanol 60:40 (20 mM Tris phosphate)

Flow rate: 1.5 mL/minute

Detection: 225 nm.

Mass spec. samples were analyzed on a MicroMass Quattro Micro mass spectrometer operated in single MS mode with electrospray ionization. Samples were introduced into the mass spectrometer using chromatography. Mobile phase for all mass spec. analyses consisted of 10 mM pH 7 ammonium acetate and a 1:1 acetonitrile-methanol mixture, column gradient conditions was 5%-100% acetonitrile-methanol over 3.5 mins gradient time and 5 mins run time on an ACE C8 3.0×75 mm column. Flow rate was 1.2 ml/min. ¹H-NMR spectra were recorded at 400 MHz using a Bruker DPX 400 instrument.

### Example A1

### Step 1: 4,6-dichloro-2-(methylsulfonyl)pyrimidine

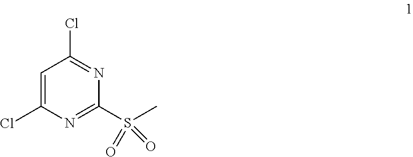

To a solution of 4,6-dichloro-2-(methylthio)pyrimidine (25 g, 0.13 mol) in dichloromethane (500 ml) at 0° C. was added m-chloroperbenzoic acid (74 g, 0.33 mol) over a period of 40 minutes. The solution was allowed to warm up to room temperature and stirred for a further 4 hours. The mixture was diluted with dichloromethane (750 ml) and then treated with 50% Na₂S₂O₃/NaHCO₃ solution, a saturated sodium bicarbonate solution and brine. The organic layer was dried over magnesium sulfate and concentrated in vacuo to afford the title compound as a white solid (26.75 g, 91% yield).

¹H NMR (DMSO D⁶, 400 MHz) δ 3.44 (3H, s), 8.43 (1H, s); MS (ES⁺) 229.

### Step 2: N-(4-(4,6-dichloropyrimidin-2-ylthio)phenyl)-3,3,3-trifluoropropanamide

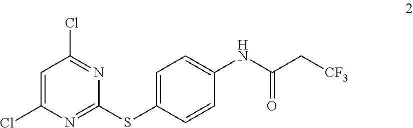 [SMILES: O=C(CC(F)(F)F)Nc1ccc(Sc2nc(Cl)cc(Cl)n2)cc1]

A solution of 4,6-dichloro-2-(methylsulfonyl)pyrimidine (8 g, 35 mmol) and 3,3,3-trifluoro-N-(4-mercaptophenyl)propanamide (8.7 g, 37 mmol) in acetonitrile (250 ml) was cooled down to −10° C. Triethylamine (4.9 ml, 35 mmol) was added dropwise over 20 minutes while maintaining the temperature at −10° C. Once added, the solution was stirred at that temperature for a further 20 minutes then allowed to warm up to room temperature and concentrated to 150 ml. Water (250 ml) was added to the reaction mixture. A solid was collected by filtration and dried by suction. This orange solid was slurried in a minimal amount of ethyl acetate. An off white solid was collected by filtration and dried in vacuo. The process was repeated to yield more solid. The batches were combined to give the desired compound (7.9 g, 56% yield). ¹H NMR (DMSO D⁶, 400 MHz) δ 3.59 (2H, q), 7.59 (2H, d), 7.70 (2H, d), 7.74 (1H, s), 10.58 (1H, s); MS (ES⁺) 383.

Other benzenethiols may be used in place of 3,3,3-trifluoro-N-(4-mercaptophenyl) propanamide in this reaction. Methods for making benzenethiols are known to one of skill in the art. Applicants have provided a few examples of benzenethiol intermediates herein (see examples S1 to S3).

### Step 3: N-(4-(4-chloro-6-(3-methyl-1H-pyrazol-5-ylamino)pyrimidin-2-ylthio)phenyl)-3,3,3-trifluoropropanamide

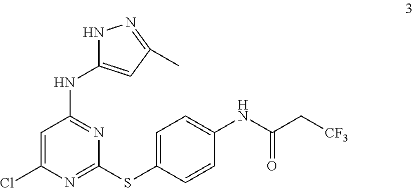 [SMILES: Cc1cc(Nc2cc(Cl)nc(Sc3ccc(NC(=O)CC(F)(F)F)cc3)n2)[nH]n1]

A solution of N-(4-(4,6-dichloropyrimidin-2-ylthio) phenyl)-3,3,3-trifluoro propanamide (14.2 g, 37 mmol), 3-amino-5-methylpyrazole (4 g, 41 mmol), sodium iodide (6.1 g, 41 mmol) and diisopropylethylamine (19.3 ml, 0.11 mol), in dimethylformamide (130 ml) was heated at 90° C. for 18 hours. The reaction mixture was concentrated to dryness. The residue was redissolved in ethyl acetate, washed with a saturated sodium bicarbonate aqueous solution and brine. The organic layer was dried over magnesium sulfate and concentrated in vacuo to afford an orange foam. The residue was slurried in dichloromethane and sonicated for 20 minutes. A solid was collected by filtration. This process was repeated to give more pure product. The pure batches were combined to give the desired product as a pale yellow solid (11.77 g, 72% yield).

¹H NMR (DMSO D⁶, 400 MHz) δ 1.96 (3H, s), 3.56 (2H, q), 5.26 (1H, br s), 6.49 (1H, br s), 7.59 (2H, d), 7.74 (2H, d), 10.21 (1H, br s), 10.57 (1H, br s), 11.90 (1H, br s); MS (ES⁺) 443.

### Step 4

The compound of formula 3 is combined with NHR₁R₂ according to methods known to one of skill in the art to provide compounds of formula I. For example, the compound of formula 3 can be heated with excess NHR₁R₂ in a suitable solvent (such as dioxane) either in a microwave or in a traditional heat bath, until completion to afford compounds of formula I.

These NHR₁R₂ amines used in the preparation of compounds of formula I are either commercially available, described in the literature (See Palmer, J. T.; et al. J. Med. Chem., 2005, 48, 7520 for the synthesis of tert-butyl-piperidin-4-yl-amine), or can be prepared according to procedures similar to the ones described herein.

### Amines Intermediates:

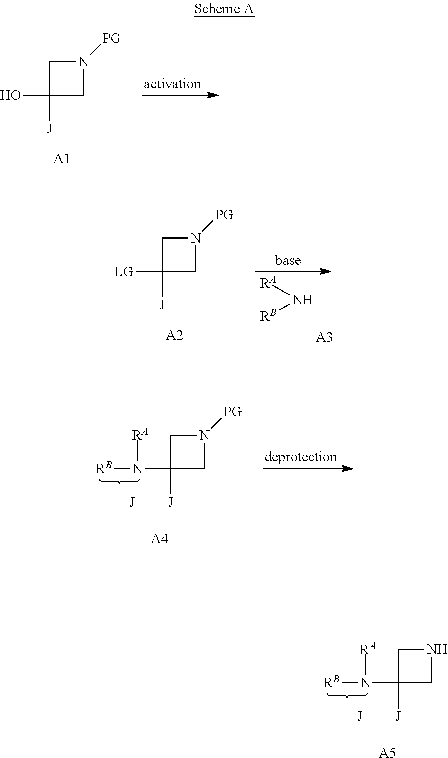

Scheme A above shows a general route for the preparation of N-substituted azetidines wherein at least one J group is bonded to the azetidine via a nitrogen atom. Protected azetidine A1 is activated with a suitable leaving group under suitable conditions to form azetidine A2, which, upon treatment with NHR^{A}R^{B} (A3) under basic conditions, forms the amine-substituted azetidine A4. Azetidine A4 is then deprotected under suitable nitrogen deprotection conditions to form compound A5.

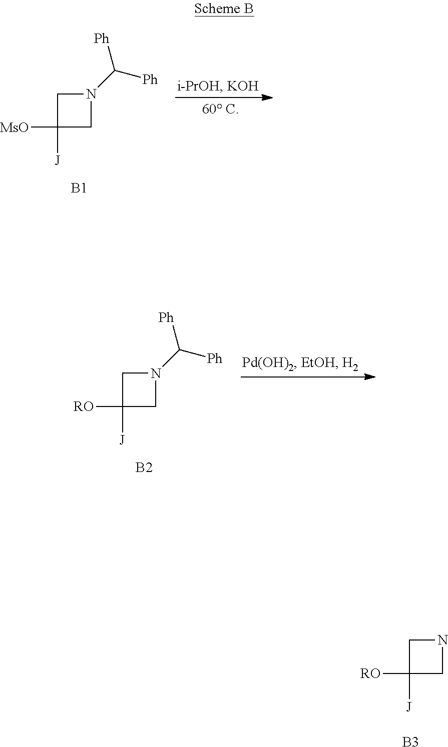

Scheme B above shows a general route for the preparation of O-substituted azetidines wherein at least one J group is OR wherein R is H or C₁₋₆alkyl.

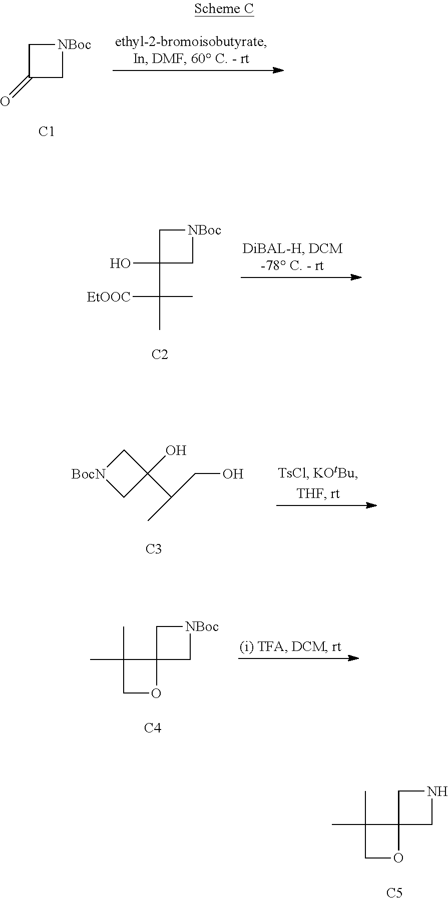

Scheme C depicts a general route for the preparation of 4-membered spirocyclic azetidines. The protected azetidinone C1 is combined with ethyl-2-bromoisobutyrate to form compound C2. Compound C2 is then deprotected with DiBAL to form compound C3. Compound C3 is then cyclized under suitable conditions to form the spirocyclic azetidine C4. Compound C4 is then deprotected under standard conditions to form compound C5.

### Example A2

### 2-methyl-2,8-diazaspiro[4.5]decane hydrochloride

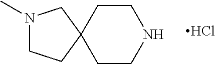

### Step 1: tert-butyl 2-methyl-1-oxo-2,8-diazaspiro[4.5]decane-8-carboxylate

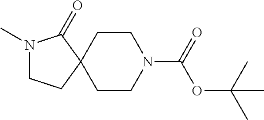 [SMILES: CN1CCC2(CCN(C(=O)OC(C)(C)C)CC2)C1=O]

A solution of 4-spiro-[3-(N-methyl-2-pyrrolidinone)]-piperidine hydrochloride (1.0 g, 5 mmol), di-tert-butyl dicarbonate (1.4 g, 6 mmol) and triethylamine (1.7 ml, 12 mmol) in dichloromethane (20 ml) was stirred at room temperature for 18 hours. The reaction mixture was diluted with dichloromethane, washed with a saturated aqueous solution of sodium bicarbonate and brine. The organic layer was dried over magnesium sulfate and concentrated under reduced pressure. The residue was purified on silica gel by flash column chromatography to afford the desired compound (1.3 g, 99% yield).

¹H NMR (DMSO D⁶, 400 MHz) δ 1.26-1.35 (2H, m), 1.40 (9H, s), 1.52 (2H, dt), 1.91 (2H, t), 2.72 (3H, s), 2.83-2.98 (2H, m), 3.27 (2H, t), 3.77-3.87 (2H, m).

### Step 2: tert-butyl 2-methyl-2,8-diazaspiro[4.5]decane-8-carboxylate

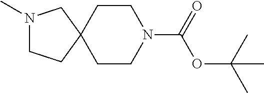 [SMILES: CN1CCC2(CCN(C(=O)OC(C)(C)C)CC2)C1]

tert-Butyl 2-methyl-1-oxo-2,8-diazaspiro[4.5]decane-8-carboxylate (1.3 g, 4.84 mmol) was taken up in tetrahydrofuran (25 ml) and cooled down to 0° C. Borane 1 M in tetrahydrofuran (15 ml, 15 mmol) was added dropwise. The reaction mixture was then heated to reflux for 18 hours. The reaction was cooled down to 0° C., quenched with methanol (15 ml), and concentrated in vacuo to give the desired compound (1.23 g, quantitative yield). ¹H NMR (CD₃OD, 400 MHz) δ 1.47 (9H, s), 1.50-1.60 (4H, m), 1.74 (2H, t), 2.37 (3H, s), 2.49 (2H, s), 2.66 (2H, t), 3.30-3.50 (4H, m).

### Step 3: 2-methyl-2,8-diazaspiro[4.5]decane hydrochloride

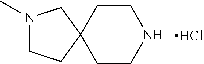

2-methyl-2,8-diazaspiro[4.5]decane hydrochloride was prepared from tert-butyl 2-methyl-2,8-diazaspiro[4.5]decane-8-carboxylate via acidic de-protection conditions known to one of skill in the art (e.g., stirring in 1.25M HCl in MeOH at room temperature for 3 h and then concentrating in vacuo to afford the desired product).

### Benzenethiols:

### Example S1

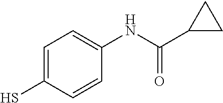 [SMILES: O=C(Nc1ccc(S)cc1)C1CC1]

### N-(4-mercaptophenyl)cyclopropanecarboxamide

Triethylamine (160.6 ml, 1.14 mol) was added to a solution of 4-aminothiophenol (65.02 g, 520 mmol) in tetrahydrofuran (1 L) cooled down to 0° C. Cyclopropanecarboxylic acid chloride (103.7 ml, 1.14 mol) was added dropwise to keep the temperature below 10° C. The reaction mixture was stirred at 0° C. for 20 minutes then warmed up to room temperature for 1 hour. The solid was filtered off and the filtrate was concentrated in vacuo.

The residue was treated with sodium hydroxide (65.02 g, 1.63 mol) in ethanol (375 ml) and water (625 ml). The reaction mixture was heated to 100° C. for 1 hour, filtered and concentrated under reduced pressure. The residue was diluted with water and filtered through a path of celite. The filtrate was acidified with concentrated hydrochloric acid and the resulting solid was filtered. The solid was dissolved in ethyl acetate (3.75 L) and washed with brine. The organic phase was dried over magnesium sulfate and concentrated in vacuo to afford the title compound (86.3 g, 86% yield). ¹H NMR (DMSO D⁶, 300 MHz) 0.76-0.85 (4H, m), 1.76 (1H, m), 5.19 (1H, s), 7.23 (2H, d), 7.5 (2H, d), 10.18 (1H, s); MS (ES⁺) 194.

### Example S2

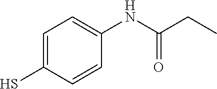

### N-(4-mercaptophenyl)propionamide

### Step 1: N,N′-(4,4′-disulfanediylbis(4,1-phenylene))dipropionamide

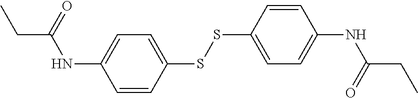 [SMILES: CCC(=O)Nc1ccc(SSc2ccc(NC(=O)CC)cc2)cc1]

Propionyl chloride (18.3 ml, 0.21 mol) was added to a solution of bis-(4-aminophenyl)disulfide (26 g, 0.10 mmol) and triethylamine (42 ml, 0.30 mol) in dichloromethane (600 ml) cooled down to 0° C. The reaction mixture was stirred at 0° C. for 5 minutes then warmed up to room temperature for 1 hour. During this time, a white precipitate formed. The reaction mixture was concentrated to half of the volume and the white solid was filtered off and washed with a small amount of dichloromethane. The filtrate was again partially concentrated and the remaining white solid was filtered off and washed. The 2 batches of solid were combined (32.4 g, 90% yield). MS (ES⁺) 361, (ES⁻) 359.

### Step 2: N-(4-mercaptophenyl)propionamide

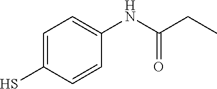

Tris-(2-carboxyethyl)phosphine hydrochloride (TCEP.HCl, 3.66 g, 12.77 mmol) was added to a solution of N,N′-(4,4′-disulfanediylbis(4,1-phenylene))dipropionamide (4 g, 11.1 mmol) and triethylamine (1.67 ml, 11.99 mmol) in a mixture of water (4 ml) and dimethylformamide (25 ml) cooled down to 0° C. The reaction mixture was allowed to warm up to room temperature and was stirred at room temperature for 90 minutes. The reaction mixture was diluted with water (100 ml), causing the precipitation of the desired product. The white solid was isolated by filtration and washed with water. The solid was dissolved in ethyl acetate, dried over magnesium sulfate and concentrated in vacuo to afford the title compound as a white solid (3.13 g, 78% yield). ¹H NMR (DMSO D⁶, 400 MHz) 1.07 (3H, t), 2.29 (2H, q), 5.24 (1H, s), 7.21 (2H, d), 7.48 (2H, d); MS (ES⁺) 182, (ES⁻) 180.

### Example S3

### 3,3,3-trifluoro-N-(4-mercaptophenyl)propanamide

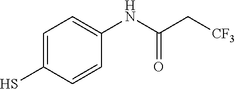 [SMILES: O=C(CC(F)(F)F)Nc1ccc(S)cc1]

### Step 1: S-4-(3,3,3-trifluoropropanamido)phenyl 3,3,3-trifluoropropanethioate

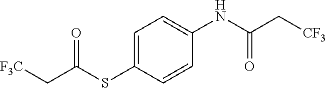 [SMILES: O=C(CC(F)(F)F)Nc1ccc(SC(=O)CC(F)(F)F)cc1]

4-Aminothiophenol is melted and charged to a flask. Degassed EtOAc (1950 mL) was added. A solution of K₂CO₃ (92 g, 670 mmol) in degassed H₂O (1300 vol) was then added. The solution was cooled to 0° C. and the 3,3,3-trifluoropropanoyl chloride (55.2 g, 600 mmol) was slowly added to keep the temperature below 10° C.). The reaction was then warmed to room temperature. The organic layer was separated and washed with brine (1300 mL). The organic layer was then concentrated on the rotary evaporator. The solid was slurried in Heptane/EtOAc (390 mL/390 mL) for 30 min. Heptane (780 mL) was then added and the slurry was cooled to 0° C. for 30 min. The slurry was filtered and the filter cake was dried under vacuum to give the desired compound (51.3 g, 87.2%).

### Step 2: 3,3,3-trifluoro-N-(4-mercaptophenyl)propanamide

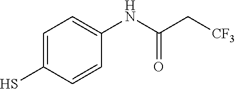 [SMILES: O=C(CC(F)(F)F)Nc1ccc(S)cc1]

S-4-(3,3,3-trifluoropropanamido)phenyl 3,3,3-trifluoropropanethioate (44.8 g, 189 mmol) and EtOH (70 mL) are charged to a flask. Concentrated HCl (22.5 mL) is slowly added to keep the temp below 30° C. The reaction is then heated to 50° C. for 17.5 h. The reaction mixture is reduced to 41 mL by vacuum distillation at 50° C. Cool the reaction to room temperature and H₂O (51 mL) is added. The slurry is filtered and the filter cake is washed with H₂O (3×35 mL). The solid is dried under vacuum to produce the desired compound (19.9 g, 58%).

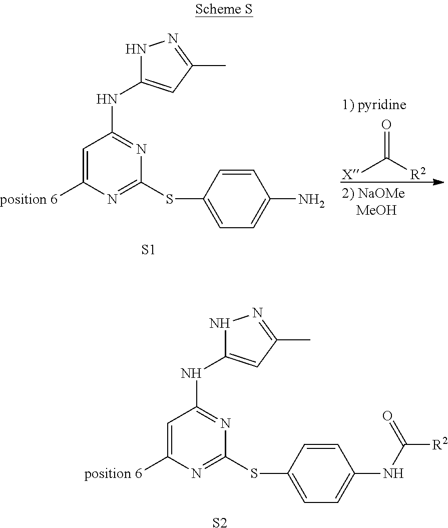

Scheme S above shows a general route for the preparation of compounds of formula I wherein R¹ is NHC(O)R². The compound of 51 is combined with a suitable acid chloride (wherein X″ is Cl) in the presence of pyridine to form an intermediate compound that, upon mixing in the presence of sodium methoxide and methanol, forms the compound of formula S2. In some embodiments, X″ can be OH, in which case a suitable acid coupling reagent is used to couple the acid to the amine Examples of suitable acid coupling reagents include, but are not limited to, EDC, DCI, and HOBT. Suitable solvents for these coupling reactions include, but are not limited to, THF, CH₂Cl₂, and dioxane.

Table 2 below depicts data for certain exemplary compounds made according to the methods described in the references, schemes, and examples provided herein. Compound numbers correspond to those compounds depicted in Table 1.

TABLE 2

| Compound | M + 1 | | LCMS |
|---|---|---|---|
| No | (obs) | 1H NMR | Rt (mins) |
| 1 | 481.3 | 1.09 (3H, t), 1.35-1.37 (2H, m), 1.44-1.46 | |
| | | (4H, m), 2.03 (3H, s), 2.26 (6H, m), 2.33 | |
| | | (2H, q), 3.13 (2H, m), 5.45 (1H, s), 5.84 | |
| | | (1H, br s), 6.75 (1H, br s), 7.46 (2H, d), | |
| | | 7.68 (2H, d), 9.05 (1H, s), 10.05 (1H, s), | |
| | | 11.65 (1H, br s) | |
| 2 | 496 | (DMSO) 1.01 (9 H, s), 1.09 (3 H, t, J 7.5), | 3.18 |
| | | 2.00 (3 H, s), 2.34 (2 H, q, J 7.5), 2.50 | |
| | | (masked signal), 3.35 (masked signal), 5.42 | |
| | | (1 H, br s), 6.01 (1 H, br s), 7.47 (2 H, d, J | |
| | | 8.5), 7.70 (2 H, d, J 8.5), 9.20 (1 H, br s), | |
| | | 10.08 (1 H, br s), 11.70 (1 H, br s) | |
| 3 | 507.4 | (DMSO) 0.82 (4H, m), 1.01 (9H, s), 1.83 | 3.24 |
| | | (1H, m), 2.03 (3H, s), 2.50 (masked signal), | |
| | | 3.35 (masked signal), 5.42 (1H, brs), 6.05 | |
| | | (1H, brs), 7.48 (2H, d), 7.70 (2H, d), 9.20 | |
| | | (1H, brs), 10.38 (1H, brs), 11.69 (1H, brs) | |
| 4 | 454.2 | (DMSO) 1.02 (9H, s), 2.09 (3H, s), 3.21- | 3.54 |
| | | 3.41 (8H, masked signals), 3.80 (3H, s), | |
| | | 5.50 (1H, s), 6.04 (1H, brs), 7.00 (1H, m), | |
| | | 7.19 (2H, m), 7.39 (1H, m), 9.25 (1H, brs), | |
| | | 11.74 (1H, brs). | |
| 5 | 535 | (d6-DMSO, 400 MHz) 1.01 (9H, s), 1.52- | 3.62 |
| | | 1.72 (8H, m), 1.82-1.91 (3H, m), 1.99- | |
| | | 2.00 (4H, m), 2.76-2.83 (1H, m), 5.39 (1H, | |
| | | s), 5.95 (1H, brs), 7.47 (2h, d), 7.75 (2H, d), | |
| | | 9.22 (1H, s), 10.09 (1H, s), 11.68 (1H, brs | |
| 6 | 505 | 1H NMR (MeOD): 1.2-1.3 (3H, t), 1.65- | 3.38 |
| | | 1.70 (6H, s), 2.20 (3H, s), 2.45-2.50 (2H, | |
| | | qd), 3.40-3.50 (5H, m), 3.80-3.95 (4H, br | |
| | | s), 5.70 (1H, s), 5.95 (1H, s), 7.70 (4H, m). | |
| 7 | 507 | (DMSO) 1.05-1.15(3H, t, Et), 1.4-1.5 (2H, | 2.91 |
| | | m, alk), 1.75-1.9 (2H, m, alk), 1.9-2.1 (7H, | |
| | | m, alk), 2.3-2.4 (2H, q, Et), 2.7-2.9 (2H, m, | |
| | | alk), 3.0-3.15 (2H, m, alk), 3.35 (H, m, alk), | |
| | | 3.5-3.6 (2H, m, alk), 4.1-4.2 (2H, m, alk), | |
| | | 5.4 (H, s, ar), 6.1 (H, s, ar0, 7.45 (2H, d, ar), | |
| | | 7.7 (2H, d, ar), 9.3 (H, s, NH), 9.5 (H, brs, | |
| | | NH) and 10.1 (H, s, NH). | |
| 8 | 511 | 1H NMR (MeOD): 1.2-1.3 (3H, t), 1.35- | 2.88 |
| | | 1.40 (6H, s), 2.20 (3H, s), 2.45-2.50 (2H, | |
| | | qd), 3.15-3.30 (3H, m), 3.65-3.70 (2H, m), | |
| | | 3.64 (2H, s), 4.40-4.50 (2H, br d), 5.70 | |
| | | (1H, s), 5.90 (1H, s), 7.60 (4H, s) | |
| 9 | 509 | 1H NMR (MeOD): 1.0-1.1 (3H, t), 1.20- | 3.59 |
| | | 1.25 (3H, t), 1.40 (6H, s), 1.70-1.80 (2H, | |
| | | qd), 2.20 (3H, s), 2.45-2.50 (2H, qd), 3.10- | |
| | | 3.30 (4H, m), 3.60-3.70 (2H, d), 4.50-4.55 | |
| | | (2H, d), 5.80 (1H, s), 5.95 (1H, s), 7.70- | |
| | | 7.80 (4H, qd). | |
| 10 | 513 | 1H NMR (MeOD): 1.20-1.25 (3H, t), 1.50 | 3.73 |
| | | (3H, s), 1.55 (3H, s), 2.20 (3H, s) 2.45-2.50 | |
| | | (2H, qd), 3.35-3.45 (5H, m), 3.85-4.00 (4H, | |
| | | m), 5.75 (1H, s), 5.80 (1H, s), 7.70-7.80 | |
| | | (4H, m). | |
| 11 | 509 | (d6-DMSO, 400 MHz) 1.10 (3H, t), 1.33 | 3.11 |
| | | (9H, s), 1.45-1.53 (1H, m), 1.91-2.01 | |
| | | (5H, m), 2.34 (2H, q), 2.89 (2H, t), 4.07 | |
| | | (2H, d), 5.43 (1H, s), 6.08 (1H, brs), 7.47 | |
| | | (2H, d), 7.71 (2H, d), 8.08 (2H, s), 9.29 | |
| | | (1H, s), 10.10 (1H, s), 11.75 (1H, brs) | |
| 12 | 464 | (DMSO) 1.98 (3 H, s), 2.33-2.26 (2 H, m), | 3.38 |
| | | 3.55 (2 H, q), 3.89 (4 H, t), 5.35 (1 H, s), | |
| | | 5.57 (1 H, br s), 7.54 (2 H, d), 7.68 (2 H, d), | |
| | | 9.37 (1 H, br s), 10.54 (1 H, s) | |
| 13 | 526.6 | (DMSO) 2.05 (3H, s), 2.33 (2H, m), 3.96 | 3.57 |
| | | (4H, m), 5.48 (1H, s), 5.60 (1H, brs), 7.58 | |
| | | (2H, d), 7.62-7.92 (6H, m), 9.54 (1H, brs), | |
| | | 10.84 (1H, brs). | |
| 14 | 510 | (d6-DMSO, 400 MHz) 0.84 (9H, s), 1.09 | 3.63 |
| | | (3H, t), 1.45 (4H, brs), 2.01 (3H, s), 2.34 | |
| | | (2H, q), 2.98-3.05 (2H, m), 3.87-3.90 | |
| | | (2H, m), 5.44 (1H, s), 6.15 (1H, brs), 7.47 | |
| | | (2H, d), 7.69 (2H, d), 9.14 (1H, s), 10.07 | |
| | | (1H, s), 11.70 (1H, s) | |
| 15 | 523 | (d6-DMSO, 400 MHz) 1.10 (3H, t), 1.37 | 3.35 |
| | | (9H, s), 1.58-1.87 (4H, m), 2.34 (2H, q), | |
| | | 2.90-2.98 (2H, m), 3.58-3.66 (1H, m), | |
| | | 3.86-3.92 (1H, m), 4.10 (1H, d), 4.20 | |
| | | (1H, d), 5.44 (1H, s), 6.04 (1H, brs), 7.48 | |
| | | (2H, d), 7.70 (2H, d), 8.26 (0.5H, brs), 8.58 | |
| | | (1H, s), 9.28 (1H, s), 10.10 (1H, s), 11.72 | |
| | | (1H, brs). | |
| 16 | 493.5 | 1.05-1.2 (3H, m, alk), 1.6-1.75 (4H, m, alk), | 3.09 |
| | | 1.85 (1H, m, alk), 2.3-2.4 (2H, m, alk), 2.8 | |
| | | (H, m, alk), 3.05 (H, m, alk), 3.2 (H, m, | |
| | | alk), 3.25-3.6 (8H, m, alk), 5.4 (H, s, ar), | |
| | | 5.8 (H, brs, ar), 7.4-7.5 (2H, m, ar), 7.7-7.8 | |
| | | (2H, m, ar), 9.15 (s, NH), 10.1 (H, s, NH) | |
| | | and 11.7 (H, brs, NH). | |
| 17 | 507.6 | DMSO 1.09 (3H, t), 1.5-1.6 (3H, m), 1.78- | 3.03 |
| | | 1.85 (1H, m), 2.03 (3H, s), 2.34 (2H, q), | |
| | | 2.84 (3H, s), 3.1-3.17 (1H, m), 3.3-3.55 | |
| | | (7H, m), 5.45 (1H, s), 6.05 (1H, s), 7.47 | |
| | | (2H, d), 7.70 (2H, d), 9.27 (1H, s), 9.80 | |
| | | (1H, brs), 10.10 (1H, brs), | |
| 18 | 533.6 | NMR (DMSO) 0.5-0.6 (2H, m, alk), 0.8-0.9 | 3.03 |
| | | (2H, m, alk), 1.05-1.15 (3H, t, CH3), 1.45- | |
| | | 1.6 (2H, m, alk), 1.75 (H, m, alk), 1.85 (H, | |
| | | m, alk), 1.95-2.1 (2H, m, alk), 2.35-2.4 (2H, | |
| | | m, alk), 2.75-2.85 (2H, m, alk), 3.0-3.15 | |
| | | (2H, m, alk), 3.35 (H, m, alk), 3.5 (2H, m, | |
| | | alk), 4.15 (2H, m, alk), 5.5 (H, s, ar), 6.15 | |
| | | (H, brs, ar), 7.5-7.55 (2H, d, ar), 7.7-7.75 | |
| | | (2H, d, ar), 9.5 (H, s, NH), 10.1 (H, s, NH) | |
| | | and 10.25 (H, brs, NH). | |
| 19 | 507.5 | 1H NMR (DMSO): 0.63 (1H, m), 1.09 (3H, | 3.00 |
| | | m), 1.28 (6H, m), 1.81 (1H, m), 2.39 (2H, | |
| | | m), 2.90-3.06 (8H, m), 3.31-3.56 (3H, m) | |
| | | 3.73 (1H, m), 5.41 (1H, s), 5.77 (1H, br s), | |
| | | 7.49 (2H, m), 7.72 (2H, m), 9.68 (1H, m), | |
| | | 10.18 (1H, s), 10.73 (1H, s) | |
| 20 | 482 | (d6-DMSO, 400 MHz) 0.90 (9H, s), 1.10 | 3.4 |
| | | (3H, t), 1.99 (3H, s), 2.33 2H, q), 3.57 | |
| | | (2H, d), 3.98 (2H, d), 5.36 (1H, brs), 5.61 | |
| | | (1H, brs), 7.49 (2H, d), 7.71 (2H, d), 9.38 | |
| | | (1H, s), 10.10 (1H, s) | |
| 21 | 466 | (400 MHz, DMSO) 0.29-0.33 (2H, m), | 3.20 |
| | | 0.40-0.49 (2H, m), 1.10 (3H, t), 1.18-1.21 | |
| | | (1H, m), 1.99 (3H, brs), 2.34 (2H, q), 3.63 | |
| | | (2H, d), 3.68 (2H, d), 5.36 (1H, s), 5.60 | |
| | | (1H, s), 7.47 (2H, d), 7.70 (2H, d), 9.21 | |
| | | (1H, brs), 10.08 (1H, s), 11.67 (1H, brs). | |
| 22 | 493.5 | DMSO) 1.08 (3H, t), 1.63 (3H, s), 1.80-2.13 | 3.26 |
| | | (7H, m), 2.37 (2H, q), 3.21 (2H, m), 3.58 | |
| | | (2H, m), 3.90 (2H, d), 4.15 (2H, d), 5.32 | |
| | | (1H, s), 5.61 (1H, brs), 7.48 (2H, d), 7.75 | |
| | | (2H, d), 9.45 (1H, s), 10.12 (1H, s), 10.57 | |
| | | (1H, s). | |
| 23 | 492 | (d6-DMSO, 400 MHz) 1.03 (9H, s), 1.10 | 4.03 |
| | | (3H, t), 2.01 (3H, s), 2.12 (2H, brs), 2.42 | |
| | | (2H, q), 3.52 (2H, t), 3.78 (2H, brs), 5.44 | |
| | | (1H, s), 5.49 (1H, s), 5.99 (1H, brs), 7.48 | |
| | | (2H, d), 7.70 (2H, d), 9.18 (1H, s), 10.06 | |
| | | (1H, s), 11.68 (1H, s) | |
| 24 | 578 | (400 MHz, DMSO) 1.09 (3H, t), 1.39 (2H, | 3.60 |
| | | brd), 2.03 (3H, s), 2.33-2.36 (4H, m), 3.15 | |
| | | (2H, brt), 3.69 (3H, s), 3.9 (2H, brs), 5.19 | |
| | | (1H, s), 5.48 (1H, brs), 6.20 (1H, vbrs), 6.96 | |
| | | (1H, dd), 7.02 (1H, dd), 7.36 (1H, dd), 7.48 | |
| | | (2H, d), 7.69 (2H, d), 9.16 (1H, brs), 10.04 | |
| | | (1H, s), 11.70 (1H, brs). | |
| 25 | 569 | (40 MHz, DMSO) 1.09 (3H, t), 1.91 (2H, | 3.70 |
| | | brt), 2.02 (3H, s), 3.31-2.37 (4H, m), 3.09 | |
| | | (2H, brt), 3.88 (3H, s), 4.22 (2H, brd), 5.46 | |
| | | (1H, brs), 6.20 (1H, vbrs), 7.01 (1H, t), 7.15 | |
| | | (1H, d), 7.33 (1H, dd), 7.37-7.39 (1H, m), | |
| | | 7.49 (2H, d), 7.70 (2H, d), 9.27 (1H, s), | |
| | | 10.06 (1H, s), 11.71 (1H, brs). | |
| 26 | 480 | (d6-DMSO, 400 MHz) 1.08 (3H, m), 1.20 | 3.26 |
| | | (6H, s), 1.98 (3H, s), 2.34 (2H, q), 3.78 (2H, | |
| | | d), 4.14-4.15 (4H, m), 5.35 (1H, s), 5.61 | |
| | | (1H, brs), 7.47 (2H, d), 7.70 (2H, d), 9.24 | |
| | | (1H, s), 10.07 (1H, s), 11.43 (1H, s) | |
| 27 | 496.5 | (DMSO) 0.95 (9 H, s), 1.10 (3 H, t), 1.70 | 3.42 |
| | | (1 H, m), 1.98 (1 H, m), 2.03 (3 H, s), 2.34 | |
| | | (2 H, q), 3.49-3.19 (4 H, masked signals), | |
| | | 5.48 (1 H, s), 5.75 (1 H, br s), 7.48 | |
| | | (2 H, d), 7.70 (2 H, d), 9.18 (1 H, br s), | |
| | | 10.04 (1 H, s). | |
| 28 | 482.5 | (DMSO) 1.11 (9H, m), 1.51 (3H, s), 2.06 | 3.54 |
| | | (3H, s), 2.40 (2H, q), 3.71-3.90 (5H, m), | |
| | | 5.45 (1H, s), 5.62 (1H, brs), 7.51 (2H, d), | |
| | | 7.78 (2H, d), 9.89 (1H, brs), 10.20 (1H, s). | |
| 29 | 494.5 | DMSO 1.15 (3H, t), 1.3-1.4 (2H, m), | 3.53 |
| | | 1.5-1.8 (6H, m), 2.02 (3H, s), 2.17-2.23 | |
| | | (1H, m), 2.42 (2H, q), 3.68 (2H, d), | |
| | | 3.82 (2H, d), 5.5 (1H, s), 5.65 (1H, s), | |
| | | 5.72 (1H, brs), 7.52 (2H, d), 7.78 (2H, d), | |
| | | 9.22 (1H, brs), 10.12 (1H, s), 11.7 (1H, brs) | |
| 30 | 468 | (400 MHz, DMSO) 0.87 (6H, d), 1.10 | 3.40 |
| | | (3H, t), 1.81 (1H, sep), 1.99 (3H, brs), 2.34 | |
| | | (2H, q), 3.61 (2h, d), 3.81 (2H, d), 5.37 | |
| | | (1H, brs), 5.47 (1H, brs), 5.63 (1H, vbrs), | |
| | | 7.47 (2H, d), 7.70 (2H, d), 9.17 (1H, brs), | |
| | | 10.05 (1H, s), 11.65 (1H, brs). | |
| 31 | 478.8 | DMSO-d6: 0.34 (2H, d), 0.40 (2H, d), 0.81 | 3.13 |
| | | (4H, d), 1.19 (1H, m), 1.81 (1H, m), 2.01 | |
| | | (3H, s), 3.66 (4H, q), 5.40 (1H, s), 5.61 (1H, | |
| | | br s), 7.48 (2H, d), 7.71 (2H, d), 9.37 (1H, | |
| | | s), 10.39 (1H, s) | |
| 32 | 496.2 | DMSO 2.03 (3H, s), 2.2-2.3 (2H, m), 3.45- | 3.32 |
| | | 3.65 (5H, m), 5.32 (0.5H, s), 5.5 (1.5H, s), | |
| | | 5.85 (1H, vbrs), 7.58 (2H, d), 7.72 (2H, d), | |
| | | 9.21 (1H, s), 10.5 (1H, s), 11.65 (1H, s) | |
| 33 | 486.3 | DMSO 1.3-1.42 (2H, m), 1.7-1.95 (8H, m), | 3.50 |
| | | 2.85-2.92 (2H, m), 3.9-4.0 (2H, m), 5.4 | |
| | | (1H, brs), 6.15 (1H, vbrs), 7.6-7.75 (3H, m), | |
| | | 7.95-8.07 (3H, m), 8.18 (1H, s), 9.20 | |
| | | (1H, brs), 11.7 (1H, brs) | |
| 34 | 445 | (d6-DMSO, 400 MHz) 0.30-0.35 (2H, m), | 3.57 |
| | | 0.37-0.43 (2H, m), 1.15-1.22 (1H, m), | |
| | | 1.59 (3H, brs), 3.66 (4H, q), 5.21 (1H, s), | |
| | | 5.58 (1H, s), 5.69 (1H, brs), 7.55-7.65 | |
| | | (3H, m), 7.96-8.00 (3H, m), 8.21 (1H, s), | |
| | | 9.19 (1H, s), 11.58 (1H, brs) | |
| 35 | 522 | 1H NMR (MeOD): 0.40-0.45 (2H, m), | 3.51 |
| | | 0.60-0.65 (2H, m), 1.3-1.4 (1H, m), 2.05 | |
| | | (2H, s), 3.25-3.40 (2H, m), 3.85-3.40 | |
| | | (4H, m), 5.40-5.50 (2H, m), 7.50-7.55 | |
| | | (2H, d), 7.65-7.70 (2H, d). | |
| 36 | 468 | 1H NMR (MeOD): 1.40-1.45 (12H, m), | 3.98 |
| | | 2.20-2.25 (3H, s) 3.05-3.20 (4H, m), 3.60- | |
| | | 3.65 (2H, m), 4.05-4.10 (2H, qd), 4.40-4.45 | |
| | | (2H, m), 5.70 (1H, s), 5.90 (1H, s), 7.00- | |
| | | 7.05 (2H, d), 7.50-7.55 (2H, d). | |

### Example 1

### Aurora-B Off-Rate and Ki* Determination

Phosphorylation of an Aurora-B peptidic substrate was measured using a radioactive-phosphate incorporation assay (Pitt and Lee, J. Biomol. Screen., (1996) 1, 47). The assay buffer consisted of a mixture of 25 mM HEPES (pH 7.5), 10 mM MgCl₂, 0.1% BSA, 10% glycerol and 1 mM DTT. Final substrate concentrations in the assay were 1.2 mM ATP (8×Km) (Sigma Chemicals) and 0.8 mM peptide (Kemptide [LRRASLG], Bachem (UK) Ltd., St. Helens, UK). Assays were carried out at 25° C. and 25 nM Aurora-B in the presence of 50 nCi/μL of [γ-³³P]ATP (Perkin Elmer, Beconsfield, UK).

Aurora-B and a DMSO stock containing the test compound were incubated in assay buffer at twenty times the final assay concentration at 25° C. for 30 minutes, prior to rapid dilution and mixture to assay buffer containing ATP and peptide constituents. Typically, final assay concentrations of the test compound ranged from 150 nM to 0 nM.

The reaction was stopped at various time-points (typically at intervals ranging from 0 to 150 minutes) by the addition of 50 μL 150 mM phosphoric acid. All assays were carried out in triplicate. A phosphocellulose 96 well plate (Millipore, Cat no. MAPHNOB) was washed with 200 μL 100 mM phosphoric acid prior to the addition of the reaction mixture (45 μL). The spots were left to soak for at least 30 minutes, prior to wash steps (4×200 μL 100 mM phosphoric acid). After drying, 100 μL Optiphase ‘SuperMix’ liquid scintillation cocktail (Perkin Elmer, Beconsfield, UK) was added to the well prior to scintillation counting (1450 Microbeta Liquid Scintillation Counter, Perkin Elmer, Beconsfield, UK).

All analysis of data was carried out using Prism 4.0 (Graphpad Software Inc.).

Ki* was determined from non-linear regression analysis of initial rate data plotted as a function of increasing inhibitor concentration. Typically, initial rate data was determined from the first 10 minutes after initiation of enzyme reaction with ATP. Data was analysed using the Morrison equation for tight-binding inhibitors (Morrison, Biochim. Biophys. Acta, (1969), 185, 269).

k_{obs} (the apparent first order rate constant of recovery of enzyme activity following initiation of enzyme reaction with substrate addition) was measured by non-linear regression analysis of enzyme activity (as measured by product concentration, [P]) plotted as a function of increasing time (t) using the equation:

$$[ P ] = {v}_{s} t + \frac{( {v}_{i} - {v}_{s} ) ( 1 - γ )}{{k}_{obs} γ} ln \{ \frac{[ 1 - γexp ( - {k}_{obs} t ) ]}{1 - γ} \}$$

where vᵢ and vₛ are the initial and steady state velocities of the reaction, and γ is given by

$$γ = \frac{{K}_{i}^{*} + [ {E}_{t} ] + [ {I}_{t} ] - Q}{{K}_{i}^{*} + [ {E}_{t} ] + [ {I}_{t} ] + Q} = \frac{[ {E}_{t} ]}{[ {I}_{t} ]} {( 1 - \frac{{v}_{s}}{{v}_{0}} )}^{2}$$

where v₀ is the initial velocity in the absence of inhibitor, Ki* is the equilibrium constant for the overall two-step binding process and [Eₜ] and [Iₜ] refer to the total concentration of enzyme and inhibitor, respectively, and

Q=[(Kᵢ*+[Iₜ]−[Eₜ])²+4(Kᵢ*[Eₜ])]^{1/2}−(Kᵢ*+[Iₜ]−[Eₜ])

(Copeland, Enzymes: A Practical Introduction to Structure, Mechanism, and Data Analysis, 2^{nd} edition (2000), Wiley-VCH, equations 10.5 to 10.7).

The mechanism of inhibition (one-step versus two-step) was determined graphically by plotting kobs as a function of increasing inhibitor concentration. For compounds that showed a two-step mechanism (non-linear relationship between kobs and [Iₜ]), the forward and reverse rate constants, k5 and k6 respectively, were determined by non-linear regression analysis of kobs versus [Iₜ] using the equation:

$${k}_{obs} = {k}_{6} + {k}_{5} \{ ( \frac{[ {I}_{t} ]}{{K}_{i}} ) / ( 1 + \frac{[ S ]}{{K}_{m}} + \frac{[ {I}_{t} ]}{{K}_{i}} ) \}$$

where Ki (=k₄/k₃) is the equilibrium constant for the formation of the initial collision complex, [S] is the substrate (ATP) concentration for which the compound is competitive and Km is the Henri-Michaelis-Menten constant for that substrate.

The overall inhibition constant, Ki*, is defined as

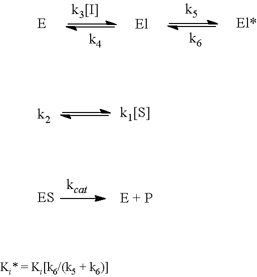

(Kapoor et al, Biochem. J., (2004), 381, 719, equations 3 and 4).

### Example 2

### Aurora-B Ki* Determination

Phosphorylation of an Aurora-B peptidic substrate was measured using a radioactive-phosphate incorporation assay (Pitt and Lee, J. Biomol. Screen., (1996) 1, 47). The assay buffer consisted of a mixture of 25 mM HEPES (pH 7.5), 10 mM MgCl₂, 0.1% BSA, 10% glycerol and 1 mM DTT. Final substrate concentrations in the assay were 1.2 mM ATP (8×Km) (Sigma Chemicals) and 0.8 mM peptide (Kemptide [LRRASLG], Bachem (UK) Ltd., St. Helens, UK). Assays were carried out at 25° C. and 25 nM Aurora-B in the presence of 50 nCi/μL of [γ-³³P]ATP (Perkin Elmer, Beconsfield, UK).

Aurora-B and a DMSO stock containing the test compound were incubated in assay buffer at twenty times the final assay concentration at 25° C. for 40 minutes, prior to rapid dilution and mixture to assay buffer containing ATP and peptide constituents. Typically, final assay concentrations of the test compound ranged from 400 nM to 0 nM.

The reaction was stopped at 0 and 10 minutes by the addition of 50 μL 150 mM phosphoric acid. All assays were carried out in triplicate. A phosphocellulose 96 well plate (Millipore, Cat no. MAPHNOB) was washed with 200 μL 100 mM phosphoric acid prior to the addition of the reaction mixture (45 μL). The spots were left to soak for at least 30 minutes, prior to wash steps (4×200 μL 100 mM phosphoric acid). After drying, 100 μL Optiphase ‘SuperMix’ liquid scintillation cocktail (Perkin Elmer, Beconsfield, UK) was added to the well prior to scintillation counting (1450 Microbeta Liquid Scintillation Counter, Perkin Elmer, Beconsfield, UK). Analysis of data was carried out using Prism 4.0 (Graphpad Software Inc.).

Ki* was determined from non-linear regression analysis of initial rate data plotted as a function of increasing inhibitor concentration. Initial rate data was determined from the first 10 minutes after initiation of enzyme reaction with ATP. Data was analysed using the Morrison equation for tight-binding inhibitors (Morrison, Biochim. Biophys. Acta, (1969), 185, 269).

Compounds 1-36 were found to have Ki/Ki* values of >3.

### Example 3

### Aurora-B Ki Determination

Phosphorylation of an Aurora-B peptidic substrate was measured using a radioactive-phosphate incorporation assay (Pitt and Lee, J. Biomol. Screen., (1996) 1, 47). The assay buffer consisted of a mixture of 25 mM HEPES (pH 7.5), 10 mM MgCl₂, 0.1% BSA, 10% glycerol and 1 mM DTT. Final substrate concentrations in the assay were 0.8 mM ATP (˜5×Km) (Sigma Chemicals) and 0.8 mM peptide (Kemptide [LRRASLG], Bachem (UK) Ltd., St. Helens, UK). Assays were carried out at 25° C. and 25 nM Aurora-B in the presence of 7 nCi/μL of [γ-³³P]ATP (Perkin Elmer, Beconsfield, UK).

Aurora-B, peptide and a DMSO stock containing the test compound were incubated in assay buffer at ˜two times the final assay concentration at 25° C. for up to 10 minutes, prior to initiation with assay buffer containing ATP. Typically, final assay concentrations of the test compound ranged from 10 μM to 0 μM.

The reaction was stopped at 0 and 180 minutes by the addition of 50 μL 150 mM phosphoric acid. All assays were carried out in duplicate. A phosphocellulose 96 well plate (Millipore, Cat no. MAPHNOB) was washed with 200 μL 100 mM phosphoric acid prior to the addition of the reaction mixture (45 μL). The spots were left to soak for at least 30 minutes, prior to wash steps (4×200 μL 100 mM phosphoric acid). After drying, 100 μL Optiphase ‘SuperMix’ liquid scintillation cocktail (Perkin Elmer, Beconsfield, UK) was added to the well prior to scintillation counting (1450 Microbeta Liquid Scintillation Counter, Perkin Elmer, Beconsfield, UK).

Analysis of data was carried out using Prism 4.0 (Graphpad Software Inc.).

Ki was determined from non-linear regression analysis of rate data plotted as a function of increasing inhibitor concentration. Initial rate data was determined from the first 180 minutes after initiation of enzyme reaction with ATP was analysed. Data was analysed using the Morrison equation for tight-binding inhibitors (Morrison, Biochim. Biophys. Acta, (1969), 185, 269).

### Example 4

### Aurora-2 (Aurora A) Inhibition Assay

Compounds were screened for their ability to inhibit Aurora-2 using a standard coupled enzyme assay (Fox et al., Protein Sci., (1998) 7, 2249). Assays were carried out in a mixture of 100 mM Hepes (pH7.5), 10 mM MgCl₂, 1 mM DTT, 25 mM NaCl, 2.5 mM phosphoenolpyruvate, 300 μM NADH, 30 μg/ml pyruvate kinase and 10 μg/ml lactate dehydrogenase. Final substrate concentrations in the assay are 400 μM ATP (Sigma Chemicals) and 570 μM peptide (Kemptide, American Peptide, Sunnyvale, Calif.). Assays were carried out at 30° C. and in the presence of 40 nM Aurora-2.

An assay stock buffer solution was prepared containing all of the reagents listed above, with the exception of Aurora-2 and the test compound of interest. 55 μl of the stock solution was placed in a 96 well plate followed by addition of 2 μl of DMSO stock containing serial dilutions of the test compound (typically starting from a final concentration of 7.5 μM). The plate was preincubated for 10 minutes at 30° C. and the reaction initiated by addition of 10 μl of Aurora-2. Initial reaction rates were determined with a Molecular Devices SpectraMax Plus plate reader over a 10 minute time course. IC50 and Ki data were calculated from non-linear regression analysis using the Prism software package (GraphPad Prism version 3.0cx for Macintosh, GraphPad Software, San Diego Calif., USA).

### Example 5

### Aurora-1 (Aurora B) Inhibition Assay (Radiometric)

An assay buffer solution was prepared which consisted of 25 mM HEPES (pH 7.5), 10 mM MgCl₂, 0.1% BSA and 10% glycerol. A 22 nM Aurora-B solution, also containing 1.7 mM DTT and 1.5 mM Kemptide (LRRASLG), was prepared in assay buffer. To 22 μL of the Aurora-B solution, in a 96-well plate, was added 2 μl of a compound stock solution in DMSO and the mixture allowed to equilibrate for 10 minutes at 25° C. The enzyme reaction was initiated by the addition of 16 μl stock [γ-³³P]-ATP solution (˜20 nCi/μL) prepared in assay buffer, to a final assay concentration of 800 μM. The reaction was stopped after 3 hours by the addition of 16 μL 500 mM phosphoric acid and the levels of ³³P incorporation into the peptide substrate were determined by the following method.

A phosphocellulose 96-well plate (Millipore, Cat no. MAPHNOB50) was pre-treated with 100 μL of a 100 mM phosphoric acid prior to the addition of the enzyme reaction mixture (40 μL). The solution was left to soak on to the phosphocellulose membrane for 30 minutes and the plate subsequently washed four times with 200 μL of a 100 mM phosphoric acid. To each well of the dry plate was added 30 μL of Optiphase ‘SuperMix’ liquid scintillation cocktail (Perkin Elmer) prior to scintillation counting (1450 Microbeta Liquid Scintillation Counter, Wallac). Levels of non-enzyme catalyzed background radioactivity were determined by adding 16 μL of the 500 mM phosphoric acid to control wells, containing all assay components (which acts to denature the enzyme), prior to the addition of the [γ-³³P]-ATP solution. Levels of enzyme catalyzed ³³P incorporation were calculated by subtracting mean background counts from those measured at each inhibitor concentration. For each Ki determination 8 data points, typically covering the concentration range 0-10 μM compound, were obtained in duplicate (DMSO stocks were prepared from an initial compound stock of 10 mM with subsequent 1:2.5 serial dilutions). Ki values were calculated from initial rate data by non-linear regression using the Prism software package (Prism 3.0, Graphpad Software, San Diego, Calif.).

| Compound | Aurora A | Aurora B |
|---|---|---|
| No | Ki (uM) | Ki (uM) |
| 1 | 0.00885 | 0.0345 |
| 2 | 0.002125 | 0.01125 |
| 3 | 0.002125 | 0.022267 |
| 4 | 0.0085 | 0.145 |
| 5 | 0.003467 | 0.021167 |
| 6 | 0.001823 | 0.014 |
| 7 | 0.0046 | 0.05025 |
| 8 | 0.002775 | 0.01535 |
| 9 | 0.0026 | 0.0193 |
| 10 | 0.00198 | 0.0225 |
| 11 | 0.0077 | 0.028 |
| 12 | 0.000707 | 0.007171 |
| 13 | 0.000382 | 0.004475 |
| 14 | 0.000595 | 0.0135 |
| 15 | 0.0025 | 0.0315 |
| 16 | 0.00575 | 0.097667 |
| 17 | 0.00205 | 0.05975 |
| 18 | 0.00275 | 0.0145 |
| 19 | 0.0054 | 0.0345 |
| 20 | 0.00035 | 0.0045 |
| 21 | 0.000387 | 0.0076 |
| 22 | 0.00068 | 0.005 |
| 23 | 0.000524 | 0.6125 |
| 24 | 0.000365 | 0.14 |
| 25 | 0.000895 | 0.23 |
| 26 | 0.000735 | 0.025 |
| 27 | 0.00035 | 0.1025 |
| 28 | 0.00135 | 0.0145 |
| 29 | 0.00067 | 0.0065 |
| 30 | 0.00115 | 0.014 |
| 31 | 0.000865 | 0.00705 |
| 32 | 0.00035 | 0.004 |
| 33 | 0.00685 | 0.064 |
| 34 | 0.00063 | 0.051 |
| 35 | 0.000354 | 0.006777 |
| 36 | 0.0052 | 0.0455 |

### REFERENCES

WO2002/057259
WO2004/000833
WO 2007/056221
WO 2007/056163
WO 2007/056164

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

The references cited herein throughout, to the extent that they provide exemplary procedural or other details supplementary to those set forth herein, are all specifically incorporated herein by reference.

While we have described a number of embodiments of this invention, it is apparent that our basic examples may be altered to provide other embodiments that utilize the compounds, methods, and processes of this invention. Therefore, it will be appreciated that the scope of this invention is to be defined by the appended claims rather than by the specific embodiments that have been represented by way of example herein.

We claim:

1. A method for selecting an Aurora B inhibitor that has cell activity comprising the steps of: 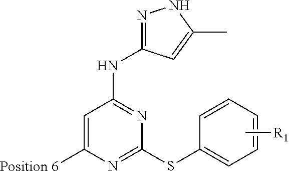
Determining Ki;
Determining Ki*; and
Selecting a compound if it has a Ki/Ki* of greater than 3.

2-7. (canceled)

8. A method for determining Ki* comprising the steps of:
Preincubating the test compound and an Aurora kinase;
Rapid dilution of the assay mixture;
Determining Ki* over a time course.

9. The method according to claim 8, wherein the time course comprises various time points at intervals from 0-150 minutes.

10. The method according to claim 8 or claim 9, wherein the final assay concentrations of the test compound ranges from 150 nM to 0 nM.

11. The method according to claim 8, wherein the initial rate data is determined from the first 10 minutes after initiation of enzyme reaction with ATP.

12. The method according to claim 11, wherein the final assay concentrations of the test compound ranges from 400 nM to 0 nM.

13. The method of claim 1, wherein Ki* is obtained according to any one of claims 8-12.

14. A compound selected by a method according to claim 1, provided that the compound is not one of the following compounds from Table 1: compound 1-2, 7, 11-22, 24-32, or 34-35.

15. The compound according to claim 14, selected from the following compounds: 3-6, 8-10, 23, or 36.

16. The compound according to claim 14, selected from the following compounds: 3-6, 8-10, 23, 33 or 36.

17. A composition comprising a compound according to any one of claims 14-16 or a pharmaceutically acceptable salt, derivative or prodrug thereof in an amount effective to inhibit an Aurora kinase and a acceptable carrier, adjuvant or vehicle.

18. The composition according to claim 17, wherein said composition is formulated for administration to a patient.

19. A method of inhibiting Aurora protein kinase activity in a biological sample comprising contacting said biological sample with a compound of any one of claims 14-16.

20. A method of treating a proliferative disorder in a patient comprising the step of administering to said patient a compound of any one of claims 14-16, or a pharmaceutically acceptable salt thereof.

21. The method according to claim 20, wherein said proliferative disorder is cancer.

22. (canceled)

23. The method according to claim 21, further comprising the sequential or co-administration of another therapeutic agent.

24. The method according to claim 23, wherein said therapeutic agent is selected from taxanes, inhibitors of bcr-abl, inhibitors of EGFR, DNA damaging agents, and antimetabolites.

25. The method according to claim 23, wherein said therapeutic agent is selected from Paclitaxel, Gleevec, dasatinib, nilotinib, Tarceva, Iressa, cisplatin, oxaliplatin, carboplatin, anthracyclines, AraC and 5-FU.

26. The method according to claim 23, wherein said therapeutic agent is selected from:
camptothecin, doxorubicin, idarubicin, Cisplatin, taxol, taxotere, vincristine, tarceva, the MEK inhibitor, U0126, a KSP inhibitor, vorinostat, Gleevec, dasatinib, and nilotinib.

27-40. (canceled)
